(19) United States
(12) Patent Application Publication
(10) Pub. No.: US 2005/0255043 A1
(43) Pub. Date: Nov. 17, 2005

(54) Bacteriophage imaging of inflammation

(76) Inventors: Donald J. Hnatowich, Brookline, MA (US); Mary Rusckowski, Southborough, MA (US)

Correspondence Address: FISH & RICHARDSON PC, P.O. BOX 1022, MINNEAPOLIS, MN, 55440-1022, US

(21) Appl. No.: 11/102,191

(22) Filed: Apr. 8, 2005

Related U.S. Application Data
(60) Provisional application No. 60/561,023, filed on Apr. 9, 2004.

(51) Int. Cl. C12Q 1/70; A61K 49/00

(52) U.S. Cl. 424/9.1; 435/5

(57) ABSTRACT

Labeled bacteriophage are disclosed which are useful for detecting a bacterial infection in vivo.

### CROSS-REFERENCE TO RELATED APPLICATION

This application claims the benefit of the filing date of U.S. Provisional Patent Application Serial No. 60/561,023, filed on Apr. 9, 2004. The contents of this prior application are hereby incorporated by reference in their entirety.

### TECHNICAL FIELD

This invention relates to the field of imaging technologies, and more particularly to the use of labeled bacteriophages to detect bacterial infections and to distinguish them from other causes of inflammation.

### BACKGROUND

Inflammation is an innate, non-specific immune response of tissues to injury. Inflammatory responses can have a variety of causes, including infection (e.g., by bacteria, viruses, and fungi), physical or chemical injury, and other physiological or pathological stimulus. Despite the variety of underlying causes, the clinical presentations of inflammatory responses can be similar. An inability to readily distinguish between different causes of inflammation has led to misdiagnoses, failures to treat with the proper antimicrobial agent, unnecessary treatments with antibiotics, treatments with unnecessarily broad spectrum antibiotics, and failures to treat non-bacterial inflammation with non-antibiotic, anti-inflammatory agents.

### SUMMARY

The invention is based, in part, on the discovery that labeled bacteriophages can be used to image bacterial infections in a subject. The bacteriophages and methods described herein can be used in a number of practical applications, e.g., diagnosing a bacterial infection, distinguishing a specific bacterial infection from a non-bacterial inflammation, identifying the type of bacteria responsible for an infection, tracking the course of a bacterial infection, and determining whether or not a treatment for a bacterial infection is effective. The use of labeled bacteriophages, e.g., radiolabeled or fluorescently labeled bacteriophages, provides a safe way to image and identify a bacterial infection in a patient in vivo.

This disclosure features methods of detecting a bacterial infection in a subject. The methods include administering to a subject an effective dose of labeled bacteriophage and imaging the labeled bacteriophage in a portion of the subject, e.g., a portion of the subject that includes a location of a suspected or diagnosed inflammation, whereby the presence of labeled bacteriophage indicates the presence of a bacterial infection. Similarly, the absence of labeled bacteriophage can indicate a non-bacterial inflammation. The methods can further include comparing the level of labeled bacteriophage that localizes to the location of the suspected or diagnosed inflammation to a control level, whereby a level of bacteriophage at the location of suspected or diagnosed inflammation that is above the control level indicates a bacterial infection. The control level can be the background level of labeled bacteriophage that localizes to a portion of the subject that does not comprise a location of a suspected or diagnosed inflammation or a control level provided by a protocol for diagnosing a bacterial infection.

This disclosure also features methods of diagnosing and treating an inflammation in a subject. The methods include performing the methods described above, wherein the presence or level of labeled bacteriophage at the site of suspected or diagnosed inflammation indicates a bacterial infection, and subsequently treating the subject with an effective amount of treatment for the bacterial infection. In cases wherein the absence or level of labeled bacteriophage at the site of suspected or diagnosed inflammation indicates a non-bacterial inflammation, the subject can be treated with an effective amount of treatment for a non-bacterial inflammation.

In other embodiments, this disclosure also features methods of identifying a type of bacterial infection in a subject. The methods include administering to a subject an effective dose of at least a first type of labeled bacteriophage that is specific for one or more first bacterial strains or species, imaging a portion of the subject, and evaluating a level of at least one of the administered labeled bacteriophages in the imaged portion of the subject. A level of labeled bacteriophage above a control level in the imaged portion of the subject indicates the presence of one or more first bacterial strains or species. Where the evaluated level of bacteriophage indicates that the subject is not infected by a first bacterial strain or species for which the first bacteriophage or bacteriophages are specific, the methods can further include administering, e.g., subsequently, to the subject an effective amount of at least one second type of labeled bacteriophage that is specific for one or more different second bacterial strains or species than the first type of labeled bacteriophage or labeled bacteriophages administered to the subject, imaging a portion of the subject, and evaluating the level of at least one second type of labeled bacteriophage. A level of the second type of labeled bacteriophage above a control level in the imaged portion of the subject indicates the presence of one or more of the different second bacterial strains or species for which the second labeled bacteriophage is specific. In some embodiments, the methods can include administering to the subject a cocktail including effective amounts of each of two or more labeled types of bacteriophage, wherein each type of bacteriophage exhibits a different range of host specificity, imaging a portion of the subject, and evaluating the level of at least one type of administered labeled bacteriophage. In these instances, a level of labeled bacteriophage above a control level in the imaged portion of the subject indicates an infection by the bacterial host of the labeled bacteriophage. When one or more labeled bacteriophage are administered in a cocktail, e.g., simultaneously, each type of bacteriophage can be differently labeled.

In some embodiments, treatments for a bacterial infection can be selected from the following possible treatments: ciprofloxacin, tetracycline, minocycline, doxycycline, erythromycin; clarithromycin, cephalosporins; amoxicillin; azithromycin; ofloxacin; ceftriaxone; and metronidazole. Treatments for a non-bacterial inflammation can exclude treatment with an antibiotic.

The methods described herein can further include performing a second imaging of the labeled bacteriophage in the portion of the subject at a later time, e.g., following administration to the subject of a second dose of an effective amount of labeled bacteriophage. The methods can further include evaluating the level of labeled bacteriophage after the first imaging and after the second imaging. In certain embodiments, a second dose of labeled bacteriophage is administered and the first dose of bacteriophage has a different label from the second dose of bacteriophage. In various embodiments, the methods include comparing the levels of bacteriophage from the first and second imagings of the subject to thereby track the course of a bacterial infection. The treatment for the bacterial infection can then be adjusted based on a comparison of the levels of bacteria at the site of infection indicated by the first and second imagings.

In another aspect, the invention includes bacteriophages conjugated with mercaptoacetyl-triglycine (MAG₃), e.g., wherein the MAG₃ is chelated to a label, e.g., a radiolabel such as Technecium-99m (^{99m}Tc), as well as bacteriophages radiolabeled with ^{99m}Tc.

In addition, the invention also features methods of imaging a bacterial infection in a subject by administering to the subject an effective dose of labeled bacteriophage and imaging the labeled bacteriophage in a portion of the subject. The label can be, e.g., a radiolabel, a fluorescent label, or a contrast agent.

In another aspect, the invention also features kits that include a labeled bacteriophage and instructions for using the bacteriophage in methods of non-invasively imaging or detecting a bacterial infection in an subject.

The kits can include N-hydroxysuccinimidyl S-acetylmercaptoacetyl-triglycine (NHS-MAG₃) and instructions for conjugating the S-acetyl NHS-MAG₃ to a bacteriophage. The kits can also provide instructions for conjugating MAG₃ conjugated bacteriophage to a label, e.g., a radiolabel, and instructions for using the bacteriophage in any of the methods described herein of non-invasively detecting a bacterial infection in a subject.

A “non-bacterial inflammation” is any inflammation that is not caused by bacterial infection. Non-bacterial inflammations, as used herein, include inflammations caused by fungi and viral agents. Non-bacterial inflammations herein also refer to inflammations that are not caused by an infectious agent.

A “subject” can be a human or an animal, e.g., a mammal such as a mouse, rat, guinea pig, hamster, dog, cat, pig, horse, goat, cow, monkey, or ape.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1 is a bar graph depciting labeled phage activity in serum or buffer remaining at the origin over time (hours) in either an ITLC-SG chromatography/acetone system or in a paper chromatography/saline system.

FIG. 2 is a bar graph depicting the percentage of radiolabeled phage binding to bacteria in vitro. E. coli 2537 (black bars), E. coli 25922 (white bars) and S. aureus (hatched bars).

FIG. 3 is a graph of percent activity remaining in the supernatant after incubation of radiolabeled phage with E. coli (open circles) and the percent activity remaining in the same supernatant after filtration through 2 μm filter (closed circles).

FIG. 4 is a C-18 HPLC radiochromatogram of the filtrate from 2 μm filtration of the supernatant remaining after incubation of radiolabeled phage with E. coli (Supernatant). Also shown are reference profiles for ^{99m}Tc-pertechnetate (^{99m}TcO₄) and ^{99m}Tc-MAG₃.

FIG. 5 is a histogram of radioactivity levels that accumulated in organ tissues harvested from normal mice injected with radiolabeled phage. Radioactivity was measured as a percent of the injected dose per organ harvested at the indicated time (hours).

FIG. 6 is a bar graph comparing the radioactivity in the infected thigh (black) and the inflamed thigh (white) of mice infected with the indicated bacterial preparations.

FIGS. 7A-7F are a series of whole body images of mice at 3 hours following administration of radiolabeled phage. Under each of the indicated bacterial strains: FIGS. 7A, 7C, and 7E are images of mice infected with a live bacterial preparation (infection-inflammation model), and FIGS. 7B, 7D, and 7F are images of mice injected with a sterilized preparation of the same (inflammation model).

FIGS. 8A and 8B are bar graphs depicting the level of radioactivity bound to bacteria in the presence or absence of Tween®20. FIG. 8A depicts the levels of radioactivity bound from ^{99m}Tc-MAG₃-E79 phage specific for Pseudomonas sp. FIG. 8B depicts the levels of radioactivity bound from ^{99m}Tc-MAG₃-P22 phage specific for Salmonella sp.

FIGS. 9A and 9B are bar graphs depicting the level of radioactivity bound to bacteria. FIG. 9A depicts the levels of radioactivity bound from ^{99m}Tc-MAG₃-VD-13 phage specific for Enterococcus sp. FIG. 9B depicts the levels of radioactivity bound from ^{99m}Tc-MAG₃-phage 60 specific for Klebsiella sp.

Like reference symbols in the various drawings indicate like elements.

### DETAILED DESCRIPTION

The bacteriophages described herein can be used in methods for distinguishing a specific type or types of bacterial infection from a non-bacterial inflammation in an subject. Labeled bacteriophages are used in the following applications disclosed herein: to image bacterial infections, to identify the specific type of a bacterial infection, to track the course of a bacterial infection, to determine the appropriate treatment for an inflammatory response, and to adjust the treatment of a bacterial infection. In some applications labeled bacteriophages are used as a probe for bacteria to determine the presence, absence, increase, or decrease of bacteria in an subject. Also disclosed herein are kits to be sold for the purpose of practicing the methods described below.

Bacteriophages

Bacteriophages possess a number of features that make them attractive for use as diagnostic agents of bacterial infections. Bacteriophages show no specificity for mammalian cells and infect bacterial cells exclusively. The administration of clean bacteriophage preparations have been reported to produce only benign consequences in animals and humans. Consequently, bacteriophages are presumed to be non-toxic (Sulakvelidze et al., Antimicrobial Agents and Chemotherapy, 45:649-659 (2001)). Furthermore, most bacteriophage strains are highly specific for a narrow range of bacterial host strains or species. Therefore, bacteriophages can safely be used as specific indicators of the presence or absence of specific bacterial strains in both animal and human patients.

Their narrow host range prevents most bacteriophages from interfering with the intestinal flora of a patient. A narrow bacteriophage host range also means that bacteriophages can be used to distinguish between different infectious bacterial strains or species, because a phage will not bind to and infect bacteria outside the phage's host range. In other words, a narrow host range allows one or more bacteriophage strains to be used as a highly specific, diagnostic tools for the identification of the specific bacterial strain(s) or species that is responsible for an infection. The ability to diagnose a bacterial strain(s) responsible for an infection allows a clinician to better tailor the treatment for the infection.

Bacteriophage Preparation and Labeling

Bacteriophages specific for over 100 genera of bacteria have been identified. See, e.g., Ackermann, Arch. Virol., 141:209-218 (1996); Ackermann, Arch. Virol., 146(5):843-57 (2001) and Table 1. Bacteriophages that are specific for particular strains can be obtained from the American Tissue Culture Collection, ATCC (Manassas, Va.). Lists of bacteriophages and their bacterial hosts, grouped by phage family, are provided in Table 1. Bacteriophages listed in Table 1 can be modified to produce long-circulating mutant bacteriophages by using or adapting the methods disclosed in Merril et al, Proc. Nat'l. Acad. Sci. USA, 93: 3188-3192 (1996). Both naturally occurring or mutant bacteriophages can be prepared or labeled using the methods described herein.

Methods of phage propagation and isolation are known in the art, e.g., Sambrook et al., Molecular cloning: a laboratory manual, 2nd ed., vol. 1, p. 66-79, Cold Spring Harbor Laboratory Press, Cold Spring Harbor, N.Y. (1989) and Merril et al., Proc. Nat'l. Acad. Sci. USA, 93:3188-3192 (1996). Typically, the appropriate bacterial host strain is grown overnight, diluted to a specific density, e.g., OD₆₀₀=0.1, and then infected with phage. Propagated phages can be harvested from lysed bacteria by centrifugation, then further purifying the phage-containing supernatant by CsCl density ultra-centrifugation and/or microfiltration (e.g., 0.22 μm filter GS filter from Millex, Millipore Corp., Bedford, Mass.). Alternatively, methods of phage preparation suitable for preparing labeled phages are described in New England Biolabs Manual, PhD-12, Phage Display Peptide Library Kit, New England Biolabs, Inc. (1999), version 2.5, pages 1-23; and in Smith and Scott, Methods in Enzymology, 217:228-257 (1993). Bacteria are grown to a specific density, diluted in buffer, infected with phage, incubated for several hours, and then centrifuged to pellet bacteria. The phage-containing supernatant is cleared by centrifugation, and bacteriophages are precipitated from the cleared supernatant, e.g., using a solution of polyethylene glycol 8000 and NaCl. Purified phages can be resuspended in a salt buffer, e.g., phosphate buffered saline (PBS).

Bacteriophages can be labeled by a number of techniques known in the art. One well-established method of radiolabeling phages involves infecting bacterial host strains with phage and growing the infected host strains in an appropriate bacterial growth medium supplemented with radiolabeled nucleotides. See, e.g., Lin et al., J. Biol. Chem., 255:10331-10337 (1980). This method results in the propagation of phages carrying radiolabeled genetic material. For some methods, however, it will be preferable to label phages using conjugation methods.

Conjugated radiolabeling of bacteriophages with radioisotopes such as Technecium-99m (^{99m}Tc) can be accomplished by the method of Hnatowich et al., J. Nucl. Med., 39:56-64 (1998). This two-step method involves conjugating purified bacteriophage with N-hydroxysuccinimidyl S-acetylmercaptoacetyl-triglycine (NHS-MAG₃) and then labeling the MAG₃-phage conjugate with ^{99m}Tc. Labeled phage conjugates are then washed and purified by polyethylene glycol precipitation. A detailed protocol for labeling phages with ^{99m}Tc is provided below in Examples 2-3.

In addition to ^{99m}Tc, conjugated radiolabeling of phages can use Indium-111, Gallium-67, or other radioisotopes suitable for nuclear imaging. Preferred chelators (instead of MAG₃) for use with these radioisotopes include diethylenetriaminopentaacetate (DTPA), 1,4,7,10-tetraazacyclododecane′-N,N′N″,N′″-tetracetic acid (DOTA), derivatives of DOTA and DTPA, as well as other chelators. See U.S. Pat. Nos. 5,155,215, 5,087,440, 5,219,553, 5,188,816, 4,885,363, 5,358,704, 5,262,532, and Meyer et al., Invest. Radiol. 25: S53 (1990).

Other methods of labeling phage particles use amine-reactive conjugates, e.g., N-hydroxysuccinimide esters other than NHS-MAG₃, or isothiocyanates of fluorescent labels that are reacted with protein amino groups of the phage particle, or maleimide groups of dyes can be reacted with protein sulthydryl groups on the phage particle. Labels suitable for conjugating to a phage particle include radioisotopes, fluorescent labels, or contrast agents. Fluorescent labels include near-infrared fluorophores such as Cy5™, Cy5.3™, Cy5.5™, and Cy7™ (Amersham Piscataway, N.J.), Alexa Fluor® 680, Alexa Fluor® 700, and Alexa Fluor® 750, (Molecular Probes Eugene, Oreg.), Licor NIR™, IRDye38™, IRDye78™, and IRDye80™, (LiCor Lincoln, Nebr.), or LaJolla Blue™, (Diatron, Miami, Fla.) and indocyanine green and the fluorochromes disclosed in U.S. Pat. No. 6,083,875.

Other labels that can be used to image bacteriophages in vivo include contrast agents. Contrast agents are useful to enable or enhance the imaging of labeled bacteriophages using imaging methods such as X-rays, computerized tomography, or Magnetic Resonance Imaging (MRI), nuclear imaging or ultrasound. For example to image bacteriophages using MRI, bacteriophages may be conjugated to any of a number of existing or novel paramagnetic nanoparticle contrast agents. The conjugation of MRI contrast agents, e.g., gadolinium, has been described, e.g., Flacke et al., Circulation, 104:1280-1285 (2001) and Allen and Meade, J. Biol. Inorg. Chem., 8: 746-750 (2003).

Candidates for Administration of Bacteriophage

Bacteriophages can be administered to animals or persons suffering from a suspected bacterial infection. Symptoms that indicate a suspected bacterial infection are known, and vary depending on the infected subject and the type of bacterial infection. See, e.g., Baron, S., ed., Medical Microbiology, 4^{th} ed., University of Texas Medical Branch (Galveston, Tex. 1996); Gorbach et al., Infectious Diseases, 3^{rd} ed., Lippincott Williams & Wilkins Publishers (Philadelphia, Pa. 2004); Pathogenesis of Bacterial Infections in Animals, 2nd edition, Iowa State University Press (Ames, Iowa 1993); The Merck Manual of Diagnosis and Therapy 17^{th} ed, Merck & Co., Inc., (Whitehouse Station, N.J. 1999) and in Aiello et al, eds., The Merck Veterinary Manual, 8^{th} Edition (Whitehouse Station, N.J. 1998). Indicators that a person or animal may harbor a bacterial infection include possible exposure to a bacterial source and/or clinical symptoms that include, but are not limited to, high fever, diarrhea, vomiting, and tissue inflammation. Suspected infections can present themselves after injury, e.g., injuries that produce cuts or open wounds. Suspected infections can also present themselves in the post-operative setting, where invasive surgical techniques are sometimes followed by incidents of “hospital infection” that can be caused by inability to maintain sterility of the operating room or of the devices inserted into the surgical patient.

Bacteriophages can also be administered to patients known to be suffering from a bacterial infection. These include, e.g., patients who are already being treated for an infection, e.g., patients suffering from sepsis, patients who have already tested positive for an infection, or patients who have otherwise been diagnosed as harboring a bacterial infection.

The following list includes the names of some common pathogenic bacteria: Streptococcus pneumoniae, Staphylococcus aureus, Streptococcus pyogenes, Haemophilus influenzae, Klebsiella pneumoniae, Pseudomonas, Pseudomonas aeruginosa, Bordetella pertussis, Clostridium (e.g., C. tetani, C. difficile, C. perfringens, and C. botulinum), Moraxella catarrhalis, Neisseria meningitides, Neisseria gonorrhoeae, Escherichia coli, Proteus, Salmonella typhii, Shigella, Yersinia, Serratia, Campylobacter, Brucella, Pasteurella, Treponema pallidium, Mycoplasma pneumoniae, Enterobacter, Treponema pertenue, Borrelia burgdorferi, Chlamydia pneumoniae, Legionella pneumophila. Additional examples of bacteria that can infect a patient are listed in the left hand columns of Table 1. Bacteriophages that are infectious towards these bacteria are listed in the right hand columns of Table 1. Bacteriophages listed in Table 1 can be modified (e.g., to make mutant long-circulating phage as in Merril et al., supra) and labeled according to the methods described herein, and subsequently used in the imaging methods described herein.

Administration of Bacteriophages

Bacteriophage preparations can be administered in many ways. Useful methods of administration to humans include: orally, in tablet or liquid forms, rectally, locally (e.g., skin, eye, ear, nasal mucosa), in tampons, rinses and creams, as aerosols or intrapleural injections and intravenously. For an extensive review of papers reporting pre-clinical administration of bacteriophage in animals and the clinical administration of phage in humans, see, e.g., Sulakvelidze et al., Antimicrobial Agents and Chemotherapy, 45:649-659 (2001).

The appropriate doses of bacteriophages vary depending on a variety of factors. Generally, doses are adjusted to ensure that labeled phage can be visualized. The ability to visualize phages will vary with factors such as the type of phage administered, the route of administration, the type of label attached to the phage, the extent of phage labeling (i.e., how much label is attached to each phage particle in the dose), the type of imaging device to be used, and the location of inflammation or suspected site of infection (e.g., deeper infections and infections of protected or denser tissues can require additional labeling). An effective dose is any dose between 10² pfu and 10¹³ pfu, e.g., 10⁵, 10⁷, or 10¹⁰ pfu.

Pharmaceutically acceptable carriers and vehicles can be used to form a composition or pharmaceutical formulation including labeled bacteriophages described herein.

Useful carriers and vehicles can include, but are not limited to, ion exchangers, alumina, aluminum stearate, lecithin, serum proteins such as albumin, buffer substances such as phosphate (e.g., PBS), glycine, sorbic acid, potassium sorbate, tris(hydroxymethyl)amino methane (“TRIS”), partial glyceride mixtures of fatty acids, water, salts or electrolytes, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polypropylene block co-polymers, sugars such as glucose, and suitable cryoprotectants.

The pharmaceutical compositions of labeled bacteriophages described herein can be in the form of a sterile injectable preparation. The possible vehicles or solvents that can be used to make injectable preparations include water, Ringer's solution, and isotonic sodium chloride solution, and 5% D-glucose solution (D5W). In addition, oils such as mono- or di-glycerides and fatty acids such as oleic acid and its derivatives can be used.

Labeled bacteriophages and pharmaceutical compositions described herein can be administered orally, parenterally, by inhalation, topically, nasally, buccally, or via an implanted reservoir. The term “parenteral administration” includes intravenous, intramuscular, intra-articular, intrasynovial, intrastemal, intrathecal, intraperitoneal, intracistemal, intrahepatic, intralesional, and intracranial injection or infusion techniques. Labeled bacteriophages can also be administered via catheters or through a needle to any tissue.

In topical uses, e.g. to check for the presence of bacteria in wounds, bums, surgical sites, epidermal or dermal inflammation sites, phage is applied in a topical composition, unbound phage is subsequently washed, and the level of bound phage remaining is subsequently determined. For ophthalmic application, the pharmaceutical compositions of the invention can be formulated as suspensions in isotonic, pH-adjusted, sterile saline.

The formulation of the conjugate can also include some other chemical compound that preserves the fluorescence properties, including, but not limited to, quantum yield, fluorescence lifetime, and excitation and emission wavelengths.

Imaging Methods

Patients can be imaged before, after, during, or both before and after administration of labeled phages. In some methods, only a portion of the patient, e.g., an arm, leg or torso, is imaged. The portion of the patient's body that is imaged includes the suspected site of infection or inflammation. Optionally, a portion of the patient's body that is believed to be uninfected and not-inflamed can also be imaged as a negative control. In some methods the imaged portion of a patient's body includes both the site of suspected infection or inflammation and also sites suspected to be uninfected and not inflamed. In some methods the patient's entire body is imaged.

Imaging devices can include magnetic resonance imaging devices (e.g., Signa Excite 3T from GE Medical Systems, Waukesha, Wis.), phosphorescent imaging devices, gamma cameras (e.g., t.cam™ Variable Camera from Toshiba American Medical Systems, Tustin, Calif.), and Near-IR CCD cameras (e.g., Cascade 512B, Photometrics, Tucson, Ariz.). When fluorescent markers are conjugated to bacteriophages, the imaging device can also include a light source capable of producing light at a specific wavelength, e.g., ultraviolet light, that causes the fluorescent marker to fluoresce.

Distinguishing Between Infection and Non-bacterial Inflammation

The phages and methods disclosed herein can be used to distinguish between non-bacterial inflammation and bacterial infections (viable and non-viable) non-invasively, i.e., without the need for biopsy or fluid extraction from a patient. By providing rapid evidence of the presence or absence of a bacterial infection, labeled bacteriophage can help clinicians make more accurate diagnosis of the causes of inflammation and avoid the needless prescription of antibiotics to treat sterile inflammations. The unnecessary prescription of antibiotics has been blamed for encouraging the development of antibiotic-resistant strains of bacteria.

The specificity of bacteriophages can make a negative result inconclusive as to whether a site of inflammation is caused by a bacterial infection, since the inflammation may be due to bacteria that are not hosts for the specific type of bacteriophages used. Thus, it can be beneficial to use a bacteriophage “cocktail” including a plurality of different types of bacteriophage, each specific for a different type or set of types of bacteria. This is discussed in more detail below.

Infections and non-bacterial inflammations can be distinguished by administering labeled bacteriophages to a patient suffering from suspected inflammation. The area of suspected inflammation is subsequently imaged to detect the presence or absence of labeled bacteriophage in the imaged area. The presence of labeled phage in the area of inflammation can be an indication that a bacterial infection is responsible for the suspected inflammation.

In some methods a bacterial infection is distinguished from a non-bacterial inflammation by quantitatively determining the levels of labeled bacteriophage that accumulate at the site of a suspected or diagnosed inflammation. A level of labeled bacteriophage at the site of inflammation that exceeds a control level is an indication that the inflamed area contains a bacterial infection, thus indicating treatment with an antibiotic directed to the bacterial host of the labeled bacteriophage. If the level of bacteriophage detected by the imaging device is below a control level, then the imaging results are an indication that the patient is not suffering from an infection by a bacterial host of the labeled bacteriophage.

Control or threshold levels of labeled bacteriophage can be determined empirically in a variety of ways. Control levels will vary with several variables, e.g., the type of bacteriophage used, the type and specific activity of the label used (e.g., the radionuclide used and the specific radioactivity of labeling), the type of tissue being imaged, the type of imaging device being used, the amount of time that has elapsed since administration of labeled bacteriophage, or a combination of these factors.

Control or threshold levels can be provided by protocols for diagnosing bacterial infections, wherein the protocols take into account the above-mentioned variables. A general method for the development of protocols for diagnosing bacterial infections includes administering different doses of labeled phage to patient populations with suspected inflammations, imaging the patients, and then correlating the level of bacteriophage at the site of inflammation with the presence, the absence, or the titer of bacteria at the site of inflammation. For the purposes of developing such a protocol, the presence, absence, or titer of bacteria at the site of inflammation is preferably determined independently, i.e., by a method other than by imaging with labeled phage. Methods for independently determining the presence, absence, or titer of bacterial infections include any bacterial diagnostic methods such as, sensitivity to antibiotics, lung x-rays, presence of bacteria-specific antibodies in the patient, or removal of tissue/fluid from the site of inflammation and subsequent identification of bacteria by a bacterial identification assay such as culturing on selective media, PCR, microscopy, or hemolysis.

Alternatively, a control level of labeled bacteriophage can be determined by comparing the level of bacteriophage that accumulates at the suspected site of inflammation to the level of bacteriophage that accumulates at an area of the patient's body that does not contain a suspected site of inflammation. Preferably, the area of the body that is not suspected of being inflamed contains similar tissue types as the site of suspected inflammation. For example, if the suspected site of inflammation is located in one limb, then the level of bacteriophage that accumulates at the suspected inflammation site is compared to the level of bacteriophage that accumulates at an anatomically similar site in the non-inflamed limb. In this manner the patient's own body provides an internal control level of non-specific labeled bacteriophage accumulation. If the level of bacteriophage that accumulates in the suspected site of inflammation is significantly higher than the control level of bacteriophage that accumulates in the non-inflamed, internal control area, then the imaging results indicate the inflammation is likely the result of a bacterial infection. The likelihood of a bacterial infection rises as more labeled bacteriophage are detected, i.e., there is a higher level of labeled bacteriophage at the site of a suspected inflammation, relative to the number (i.e., level) of bacteriophage imaged at the non-inflamed site.

Methods for imaging, analyzing, and quantifying imaging data are provided with imaging products, e.g., in the technical manual for the software packages used in conjunction with imaging devices such as CCDs or gamma camera. See also Sandler et al., Eds. Diagnostic Nuclear Medicine, Williams & Wilkins Company, Baltimore, (3^{rd} ed., 1996) and (4^{th} ed. 2002).

Identifying the Type of a Bacterial Infection

The phages and methods disclosed herein can also be used to non-invasively identify the type or the strain of bacteria responsible for an infection. Most characterized bacteriophage strains preferentially bind to and infect only a narrow range of bacterial hosts. See, e.g., Ackermann, Arch. Virol., 141:209-218 (1996); Ackermann, Arch. Virol., 146(5):843-57 (2001) and Table 1. Thus, the accumulation at the site of suspected infection of a bacteriophage strain that is specific for a particular strain or species of bacteria is an indication of infection by the particular strain or species of bacteria, for which the administered bacteriophage is specific.

For example, the accumulation of labeled bacteriophage CEV1 (a member of the T-even family), which is specific for strains of E. coli O157:H7, is an indication of infection by E. coli O157:H7. In another example, the accumulation of a labeled bacteriophage specific for Salmonella typhimurium, e.g. a labeled version of one of the Salmonella specific phages described in U.S. Pat. No. 6,699,701, is an indication of a Salmonella infection.

The ability to precisely define the presence of bacteria at the site of inflammation enables the clinician to provide targeted antibiotic treatments for the infection. Thus, the bacteriophage imaging disclosed herein can reduce the need for treating inflammations with overly broad-spectrum antibiotics. The over-prescription of broad-spectrum antibiotics has been widely blamed for the rise and spread of antibiotic resistant bacterial strains.

In one example, the phages and methods described herein can be applied to the potentially devastating problem of suspected prosthetic joint infections. Labeled bacteriophage imaging methods can be used to detect which type of bacteria, if any, are present at the site of an implanted prosthetic device, without resorting to the removal of synovial fluid or tissue biopsy. In some cases synovial fluid or tissue biopsies from the site of a suspected infection are contaminated with normal skin flora, which leads to uncertainty as to whether the joint is infected at all or whether the joint is infected with skin bacterial flora in addition to other bacteria. This uncertainty in diagnosis can lead not only to overly aggressive broad-spectrum antibiotic prescription, but also to the removal of the entire prosthetic joint, and the need to implant a new prosthesis. Thus, the phage imaging methods disclosed herein can be used to more rationally treat suspected infection of prosthetic joints by first confirming an infection is the source of inflammation, and second, diagnosing the particular strain(s) responsible for the infection. Knowledge of the bacterial strains responsible allows the clinician to treat the infection with the appropriate spectrum antibiotics.

When a specific bacterial infection is diagnosed using the methods disclosed herein, the patient can be administered a treatment specific for that bacteria. The appropriate doses and antibiotics for treating different classes of bacterial infections can be found, for example, in Beers et al, eds., The Merck Manual of Diagnosis and Therapy 17^{th} ed, Merck & Co., Inc., (Whitehouse Station, N.J. 1999) and in Aiello et al, The Merck Veterinary Manual, 8^{th} Edition (Whitehouse Station, N.J. 1998).

When the methods disclosed herein indicate that a patient is suffering from a non-bacterial inflammation and not a bacterial infection, then the patient can be administered a treatment for a non-bacterial inflammation. If the patient is being administered prophylactic antibiotic treatment before the diagnosis of a non-bacterial inflammation using the methods disclosed herein, then the antibiotic treatment can be stopped after the diagnosis of non-bacterial inflammation. Treatments for non-bacterial inflammations of different tissues and under various indications are described in Beers et al, eds., The Merck Manual of Diagnosis and Therapy 17^{th} ed, Merck & Co., Inc., (Whitehouse Station, N.J. 1999) and in Aiello et al, The Merck Veterinary Manual, 8^{th} Edition (Whitehouse Station, N.J. 1998).

Administration of Different Phages

In some applications a patient is administered more than one strain of labeled bacteriophages. Because of their narrow host specificity, it is sometimes desirable to administer more than one bacteriophage to a patient. For example, to increase the likelihood that a diagnosis of non-bacterial inflammation is correct, different labeled phage strains, each of which has a different host strain specificity can be administered to a patient. After the administration of labeled phages to the patient, the site of suspected inflammation is imaged, and the accumulation of bacteriophage is measured.

Bacteriophages can be administered in a cocktail, i.e., a mixture, of one or more different types of labeled phages. Phages that exhibit infectious specificity to different types of bacteria can also be labeled differently. Using a cocktail of differently labeled phage strains allows the level of each phage strain in the cocktail to be determined independently. If a particular type of label accumulates above a control level at the imaged site of inflammation, then a diagnosis of non-bacterial inflammation is not indicated. Instead, such a result indicates an infection by a bacterial host for the bacteriophage carrying the label that accumulates above a control.

Alternatively, different types of labeled phages administered in a cocktail can carry the same label. If the total accumulation of the label does not exceed a control, then a diagnosis of non-infection by the hosts of administered phage is appropriate. Additionally, a diagnosis of non-bacterial inflammation may be appropriate. If the total accumulation of label at the site of inflammation does exceed a control, then the diagnosis of non-bacterial inflammation is not indicated, instead a bacterial infection is indicated.

In some applications, different types of labeled phages are administered sequentially, not in a single cocktail. For example, a labeled phage specific for one type of bacterium is administered to a patient. The accumulation of phage at the suspected site of infection is measured using an imaging device. At some time after the administration of the first phage strain, e.g., 1, 2, 3, 4, 5, 6, 7, 8, 10, 12, 24, or 36 hours later, a second type of labeled phage strain is administered to the same patient. The second labeled phage strain will preferably have a different host range. The accumulation of the second type of labeled phage at the site of infection is measured using an imaging device. The accumulation of one of the phage strains, e.g., above a control level, at the site of inflammation is an indication that the inflammation is caused by an infection of bacterial host of the labeled phage strain that accumulates at the site. If neither phage strain accumulates at the site of inflammation, e.g., accumulates above a pre-selected control, then the sequential imagings indicate that inflammation is most probably not caused by a bacterial host of either of the labeled phage strains administered. In the latter case, a third labeled phage strain can be optionally administered, preferably with a different host range from the first two administered labeled phage strains, and the accumulation of the third pliage strain at the site of inflammation can be measured by the imaging methods disclosed herein. The serial administration of labeled phage can be continued through 4, 5, 6, 7, 8, 9, 10 or more administrations of labeled phage.

A phage cocktail can comprise, e.g., 2, 5, 10, or 20 different species of labeled bacteriophages specific for one or more types or species of bacteria. For example, a cocktail of labeled phages VD-13, P22, E79, and 60 can detect an infection by Enterococcus, Salmonella, Pseudomonas, or Klebsiella species. A cocktail of labeled phages 2BV, NP1, AC1, and HB-623 can detect an infection by Enterobacter, Neisseria, Staphylococcus, or Streptococcus species. The specific bacteriophage strains included in the cocktail can be designed by one skilled in the art to detect a desired subset of bacterial species.

Tracking the Course of an Infection

The bacteriophages and imaging methods provided herein can be used to track the course of a bacterial infection. After diagnosing a bacterial infection in a patient, the levels of bacteria present at a site of infection in the patient is measured using any of the labeled bacteriophage described herein. The patient may be optionally treated for the infection, e.g., with antibiotics. A dose of labeled bacteriophage that is infectious for the diagnosed bacterial strain is administered to the patient. The site of infection is imaged, and the amount of bacteria present at the site of infection is measured indirectly as a function of the level of labeled phage detected by imaging. At some time after the site of infection is first imaged, e.g., 1, 2, 4, 6, 8, 12, 24, 36, 48, 60, 72 or 96 hours after the first imaging, the site of infection is imaged again, optionally after administration of a second dose of phage to the patient. A reduction of the level of labeled phage at the site of infection, as detected by the second imaging, is an indication that the levels of viable and non-viable bacteria at the site of infection are diminishing. An absence of significant reduction in the levels of labeled phage that accumulate at the site of infection can be an indication of a persistent bacterial infection that is not being cleared.

In a different method, a patient diagnosed with an infection is administered labeled bacteriophage that is infectious for the diagnosed bacterial strain. The patient is imaged both at the site of infection and at a site that is not suspected to harbor an infection. The level of bacteriophage accumulating at the site of infection is compared to the level of bacteriophage that accumulates at the non-infected site. The amount of bacteria present at the site of infection is measured indirectly as a function of the level of labeled phage detected by imaging that is above the control level of phage measured at the non-infected site. At some time after the first imaging, e.g., 1, 2, 4, 6, 8, 12, 24, 36, 48, 60, 72 or 96 hours after the first imaging, the site of infection and the site of non-infection are imaged again, optionally after administration of a second dose of phage to the patient. A reduction in the difference of the level of bacteriophage measured at the site of infection relative to the level of bacteriophage measured at the non-infected site is an indication that levels of bacteria at the site of infection are diminishing. If the difference between the levels of bacteriophage detected at the site of infection relative to a non-infected site remains the same or increases, that can be an indication that levels of bacteria at the site of infection are not diminishing.

To track the course of the bacterial infection over time, a patient can be imaged multiple times after diagnosis and treatment for a bacterial infection. Optionally, multiple administrations of a bacteriophage specific for the infecting bacteria can be given to the patient some time before each of the multiple imagings. An observed decline in the accumulation of bacteriophage at the site of infection (alone or relative to a control non-infected site) can be an indication that the bacterial infection is being cleared. No decline, or an increase in the accumulation of phage at the site of an infection can be an indication that bacteria are not being cleared from the site of infection.

If the methods disclosed herein indicate that a bacterial infection is being cleared, then no change in the treatment of the infected patient is indicated. A decline in bacterial levels is an indication that a treatment regimen (or that the decision not to treat the patient) is effective. If the bacteria at the site of infection are not being cleared, then a change in treatment is indicated. Changes in treatment can include increasing the dose of the treatment (e.g., antibiotic) being given to the patient, or changing the treatment, e.g., switching to a different antibiotic or stopping antibiotic treatment altogether.

Kits

The labeled phages described herein can be sold in containers that include instructions for the use of the labeled phage in any of the methods disclosed herein. Other kits will include unlabeled phage and a label along with instructions for either labeling the phage, for using labeled phage in the methods disclosed herein, or for both labeling and using the labeled phage in the methods disclosed herein.

### EXAMPLES

The invention is further described in the following examples, which do not limit the scope of the invention described in the claims. These examples report the use of radiolabeled phage as a non-invasive diagnostic imaging agent for three different bacterial infections. Bacteriophage M13 was labeled with ^{99m}Tc. The binding affinity of labeled M13 to bacteria was assayed in vitro as well as in vivo using both a bacterial infection-inflammation model and a sterile inflammation model.

Starting Materials and Experimental Methods

The M13 phage and Escherichia coli (strain 2537) were obtained from New England Biolabs, Inc, (Beverly, Mass.). A second E. coli strain (25922) and Staphylococcus aureus (strain 29213) were obtained as plate cultures from the Clinical Microbiology Department at this university. LB media containing bacto-tryptone (Sigma-Aldrich, St. Louis, Mo.), yeast extract (Sigma), and NaCl was prepared according to standard procedures and autoclaved. The S-acetyl NHS-MAG₃ was synthesized in house (according to Winnard et al., Nucl Med Biol., 24:425-432 (1997)) and the structure was confirmed by elemental analysis, proton NMR and mass spectroscopy. The ^{99m}Tc-pertechnetate was eluted from a ⁹⁹Mo-^{99m}Tc generator (Perkin-Elmer, Billerica, Mass.). All other chemicals were used as supplied. The CD-1 male mice, 22-25 grams were obtained from Charles River, Wilmington, Mass.

Stocks of the three bacteria were grown and maintained as plate cultures and stored at 4° C. The day before each study, liquid cultures in LB media were seeded and grown overnight at 37° C. while rotating at 250 rpm. Bacteria were counted using a hemacytometer and diluted in LB media for use. To prepare the infection-inflammation model, aliquots of the bacterial cultures were diluted in LB media to a concentration of 4.8×10⁸/ml, and 0.1 ml was administered subcutaneously in one thigh of CD-1 mice. To prepare the sterile inflammation model, the diluted bacterial preparations (4.8×10⁸/ml) were heated in a boiling water bath for 30 minutes, spun at 8,200× g for 3 minutes (Biofuge-15, Heraeus Instruments, Germany), and then sonicated for 10 minutes. No growth was apparent one day after samples were plated on LB agar as an indication of sterility. As before, 0.1 ml of the cell free broth was administered subcutaneously in one thigh.

### Example 1

### Preparation of Phage

The M13 phage was propagated in E. coli 2537, according to the methods described in “PhD-12 Phage Display Peptide Library Kit” in New England Biolabs Manual, New England Biolabs, Inc., version 2.5; pages 1-23 (Beverly, Mass., 1999). In brief, a liquid culture of the E. coli was diluted 1:100 with 20 ml of LB media to which 10 μl of the stock phage (2×10¹¹ Plaque Forming Units (PFU)) was added. After 4.5 hours of vigorous shaking at 37° C., the sample was spun at 1,400×g (Jouan, Model CR 4.12 Jouan Inc., Winchester, Va.) for 10 minutes to pellet the bacteria. The supernatant was spun again, then transferred to a fresh tube and a solution of polyethylene glycol 8000 and 20% weight/volume (w/v) 2.5 M NaCl (PEG/NaCl) at a ratio of 1:6 volume/volume (v/v) was added, as described in Smith et al., Methods in Enzymol., 217:228-257 (1993). After the sample was spun at 15,000×g for 20 minutes, the phage pellet was recovered and suspended in Dulbecco's phosphate buffered saline (PBS, pH 7.2) (Gibco/Invitrogen Corp., Carlsbad, Calif.). The precipitation was repeated with the PEG/NaCl solution. The final phage pellet was suspended in PBS and stored at 4° C.

### Example 2

### Conjugation of the M13 Phage with MAGs

Phage particles were conjugated with S-acetyl NHS-MAG₃ following methods standard in this laboratory for the radiolabeling with ^{99m}Tc of proteins, peptides and oligomers (Hnatowich et al., J. Nucl Med. 1998;39:56-64). In brief, to 50-100 μl of PBS containing about 10¹⁰ PFU/μl of phage was added to 2-4 μl of 0.1M sodium bicarbonate solution, pH 9.0, for a final pH of 8.0, and with constant agitation, 2-4 μl of a fresh solution of NHS-MAG₃ in dry N,N-dimethyl formamide (DMF) (1 mg/ml). The volume of DMF was always less than 10% of the final volume. The conjugation mixture was incubated at room temperature for 45 minutes, then unbound MAG₃ was removed by precipitation of the phage with the PEG/NaCl solution as before and the sample spun at 15,000×g for 15 minutes at 4° C. to pellet the phage. The MAG₃-phage pellet was suspended in 50-100 Aμl PBS and purified once-again by re-precipitation with PEG/NaCl. The final pellet of conjugated phage was suspended in PBS and stored at 4° C.

### Example 3

### Radiolabeling of MAG₃-Phage with ^{99m}Tc

To radiolabel with ^{99m}Tc, an aliquot of sodium tartrate (50 mg/ml) in 0.5 M sodium bicarbonate, 0.25 M ammonium acetate, 0.175 M ammonium hydroxide buffer (pH 9.2) was added to the MAG₃-phage (5-50 μl, at a concentration ˜10⁹ PFU/μl) so that the final concentration of tartrate was 7 μg/ml in the labeling mixture. After addition of 9.25-37 MBq of ^{99m}Tc pertechnetate generator eluant, 2 μl of a fresh solution of SnCl₂.2H₂O (1 mg/ml in 10 mM HCI) was added. The labeling mixture was incubated at room temperature for 30-60 minutes. The ^{99m}Tc-labeled-MAG₃-phage was purified by precipitation twice with PEG/NaCl as described above. Radiochemical purity was estimated by Instant Thin Layer Chromatography (ITLC-SG from Gelman, Ann Arbor, Mich.) with acetone as solvent and by paper chromatography (Whatman no. 1, VWR, Boston, Mass.) with saline as solvent. Both radiolabeled phage and colloids remain at the origin in both systems, while pertechnetate, labeled tartrate, and MAG₃ migrate in saline and only pertechnetate migrates in acetone. The chromatography strips were cut into 1 cm sections and the radioactivity determined in a gamma well counter (Cobra II Auto-Gamma, Packard Instrument Co., Downers Grove, Ill.). As a control, the identical labeling procedure was performed on phage that had not been conjugated with MAG₃.

Analysis by both ITLC and paper chromatography of all radiolabeled phage preparations showed greater than 90% of the label remaining at the origin, almost certainly as labeled phage. Radioactivity binding to native phage without MAG₃ was less than 5%.

### Example 4

### In Vitro Testing of Labeled Phage Stability

To test the stability of the label on free phage, the ^{99m}Tc-labeled phage (20 μl, 4×10¹⁰ PFU) were added to 0.2 ml of fresh human serum or PBS at 37° C. and aliquots removed at 15 minutes, 30 minutes, 1 hour, 3 hours and 18 hours for analysis in duplicate by ITLC/acetone and paper/saline.

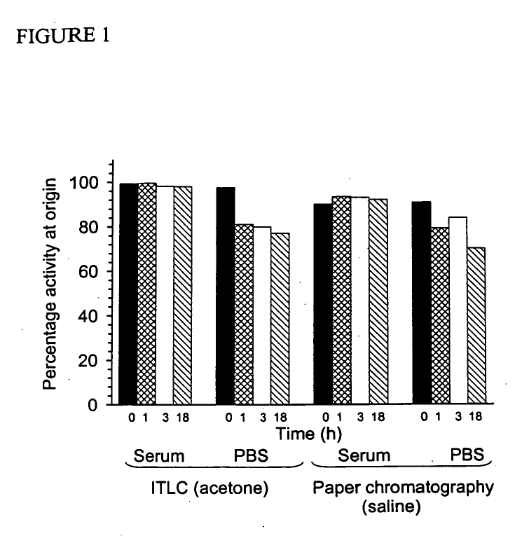

FIG. 1 presents a histogram of the activity remaining at the origin upon analysis by paper and ITLC chromatography of labeled phage in serum and buffer over time. As shown, the label on phage showed no important instabilities leading to migration in serum. At 18 hours of incubation 99% (ITLC) and 91% (paper) of the added activity still remained at the origin. The results in buffer were somewhat lower at 80% (ITLC) and 77% (paper) at 15 minutes but with minimal change over time thereafter. The radiolabeled phage were thus shown to be stable under the conditions of incubation, including serum incubation.

Binding of ^{99m}Tc-Phage to Bacteria

Binding of the labeled phage to bacteria was measured following the addition of ^{99m}Tc-phage (3.5×10⁷ PFU) to 0.5 ml of the two E. coli strains and Staphylococcus aureus each at a cell count of 8×10⁸/ml. Samples in triplicate were removed at 1 minute, 5 minutes, and 10 minutes and spun (8,200×g for 3 minutes). The bacterial pellet was washed with PBS and counted for radioactivity.

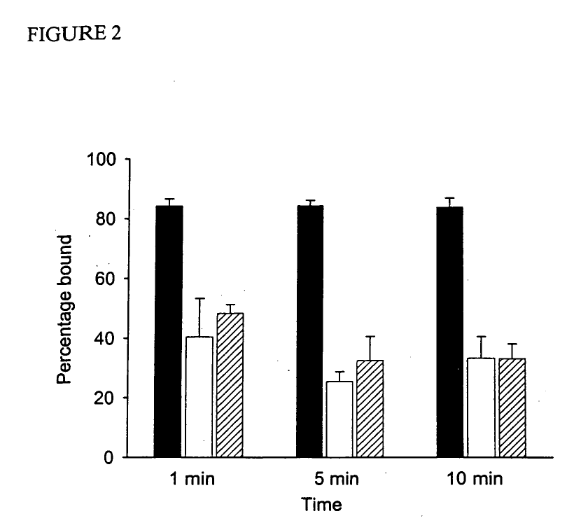

When the labeled phage were added to the live bacterial suspensions, binding was immediate as shown in FIG. 2. As early as 1 minute, 84% of the label was associated with E. coli 2537 (black bars) and was unchanged over 10 minutes. By contrast, E. coli 25922 (white bars) and S. aureus (hatched bars) bound only about 40-45% at 1 minute followed by a slight decrease to about 30%- 35% at 5 minutes and 10 minutes in both cases. Thus, even though the labeled M13 phage showed preferential binding to one E. coli strain, the phage also bound, though at a lower level, to a second E. coli strain and to S. aureus.

Binding of ^{99m}Tc-Phage to Live and Heat-killed Bacteria

To determine if the ^{99m}Tc-phage bound to heat killed bacteria, the labeled phage were incubated with both live and heat killed preparations. The live bacterial preparations were adjusted to a concentration of 5.2×10⁸/ml in LB media, each preparation was divided into two aliquots and one aliquot was heated in a boiling water bath for 30 minutes for sterilization. All six preparations of live and heat killed bacteria (0.5 ml of each) were incubated in duplicate in a 37° C. water bath with the ^{99m}Tc-phage (3.5×10⁶ PFU, about 0.011 MBq). After 5 minutes the samples were spun at 8,200×g for 3 minutes, the supernatant was removed, and the pellet was washed with PBS. The wash was pooled with the supernatant and counted in a gamma well counter along with the pellets for associated activity.

Incubation of labeled phage with live and heat killed bacteria demonstrated that the labeled phage bound almost equally to both and regardless of bacteria type. As shown in Table 2, while more radioactivity was associated with the live bacteria than with the heat killed preparations, the difference was only about 11% irrespective of the bacterial type.

TABLE 2
^{99m}Tc-phage binding to live and heat killed bacteria.
Added radioactivity in pellet and supernatant (%). Mean of N = 2.

| Bacteria | Condition | Pellet | Supernatant |
|---|---|---|---|
| E. coli 2537 | Live | 71 | 24 |
| | Heat Killed | 59 | 37 |
| S. Aureus | Live | 86 | 9 |
| | Heat Killed | 76 | 20 |
| E. coli 25922 | Live | 83 | 12 |
| | Heat Killed | 70 | 25 |

Analysis of Supernatant Radioactivity

Since heat sterilization of bacteria is believed to damage the plasma membrane such that the cell constituents can escape leaving membrane fragments (Brock, T. D., p. 206 in Biology of Microorganisms, Prentice-Hall Inc. Englewood Cliffs, N.J., 1970), it was important in this study to establish whether the radiolabeled phage was retained on bacteria once the membranes were fragmented, in this case by heat denaturation. We wished to determine whether the phage remained bound to the bacterial cell wall or membrane fragments once the infected bacteria were killed. The method of analysis consisted of two serial centrifugations, a short spin to pellet bacteria and large fragments and their associated phage followed by a second spin to pellet smaller fragments. As before, the labeled phage (10⁹ PFU, about 0.011 MBq) was added to 4 ml of live and heat killed E. coli preparations (each at 1.5×10⁹/ml) and incubated at 37° C. Samples were removed at 1 minute, 15 minutes, and 60 minutes and spun at 850×or 1 minute. The supernatants were then spun for a further 10 minutes at the same speed. The supernatants and pellets were then counted.

The level of radioactivity in the supernatant of the above preparations was investigated further for E. coli 2537 by measuring the radioactivity in the supematant after a second, longer, centrifugation. As shown in Table 3, in the case of the live bacteria, radioactivity in the supernatants was unchanged after the first short (1 minute at 850×g) and second longer (10 minutes at 850×g) centrifugation. By contrast, in the case of the heat-killed bacteria considerably more radioactivity remained in suspension after the first centrifugation, but this radioactivity was brought down after the second centrifugation. The first, short, centrifugation brought down most intact bacteria and large cell debris but not the small cell fragments generated by heat killing. Since almost all the radioactivity could be brought down by the second spin, that radioactivity must have remained associated with cell fragments. These results showed, once again, that the radiolabel was stable under the studied conditions.

TABLE 3
Radioactivity in supernatant after serial centrifugations
after time of incubation at 37° C.
Added radioactivity in supernatant (%). Mean of N = 2.

| Incubation | | First Supernatant | Second Supernatant |
|---|---|---|---|
| at 37° C. | E. coli (2537) | (1^{st} short spin) | (2^{nd} longer spin) |
| 1 minute | Live | 4 | 4 |
| | Heat Killed | 36 | 6 |
| 15 minutes | Live | 5 | 5 |
| | Heat Killed | 14 | 5 |
| 60 minutes | Live | 6 | 6 |
| | Heat Killed | 21 | 7 |

1^{st} spin . . . 1 minute at 850 × g
2^{nd} spin . . . 10 minutes at 850 × g

Further study was done on the source of radioactivity remaining in suspension after the initial (1 minute at 850×g) spin of bacteria incubated for different times with ^{99m}Tc-labeled phage. To obtain sufficient radioactivity for an HPLC analysis of the supernatant, the above procedure was repeated but only in live bacteria and using phage radiolabeled at a higher specific activity. E. coli (6 ml at 5.2×10⁸/ml) was incubated with ^{99m}Tc-phage (10⁹ PFU, about 14.8 MBq) in a 37° C. water bath as before and samples removed in duplicate at 1 minute, 30 minutes, 60 minutes and 105 minutes. To remove particulate matter, samples were first spun for 1 minute at 1,000×g and the supernatants filtered through a 0.2 μm filter (13 mm Acrodisc, Gelman Sciences, Ann Arbor, Mich.). The filtrate was analyzed by reversed phase HPLC (C-1 8, YMC-pack ODS-AMQ, 4.6×250 mm, Waters, Milford, Mass.) using a linear gradient at 1 ml/minute going from 100% eluant A (0.1% TFA in water) to 60% eluant B (0.1% TFA in 90% acetonitrile/10% water) in 30 minutes. Analysis was also performed by ITLC/acetone and paper/saline on the unfiltered samples.

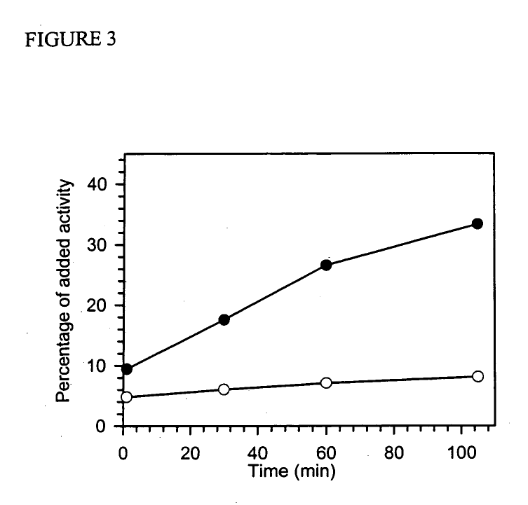

FIG. 3 shows a graph of the results of C-18 HPLC analysis of filtered supernatant (open circles) and unfiltered supernatant (closed circles) collected after incubation times indicated on the x-axis. After a short centrifugation, the unfiltered supernatant from a 1 minute incubation contained 9% of the added radioactivity, while after a 105 minutes incubation the unfiltered supernatant contained 33% of added radioactivity. The percentage of radioactivity passing through the filter (open circles), on the other hand, remained relatively constant at about 7%, even for supernatant collected after the 105 incubation. These results suggest that radioactivity removed by filtration is bound to particulate matter, almost certainly bacterial wall and membrane fragments. Thus, the increasing radioactivity observed in supernatants of longer incubations are due to increased phage-mediated cell killing, which generates phage-bound fragments that are not brought down by a short centrifugation but that are removed by filtration. The data confirm that the label on the bacteriophage was stable even after phage-mediated bacterial lysis.

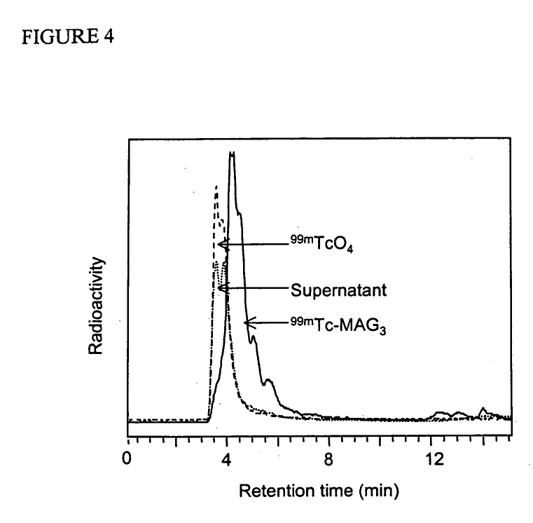

The radioactivity in the filtrate was analyzed by reversed phase C-18 HPLC. FIG. 4 presents the radiochromatogram obtained by analyzing the 30 minutes incubate along with radiochromatograms of pertechnetate and ⁹⁹mTc-MAG₃ for comparison. Each filtrate radiochromatogram showed a single peak with a retention time identical to that of pertechnetate and was therefore most probably pertechnetate. The recovery was always greater than 90%.

### Example 5

### In Vivo Testing of Labeled Phage

Biodistribution of^{99m}Tc-Phage in Normal Mice

Biodistribution of the labeled phage was measured in normal CD-1 mice. About 0.1 ml, containing 2×10⁹ PFU (about 1.18 MBq), of labeled phage were administered to normal mice through a tail vein. Animals were sacrificed at 30 minutes, 3 hours, 6 hours, and 24 hours (n=2) and organs of interest and blood were removed, weighed and counted in the gamma well counter.

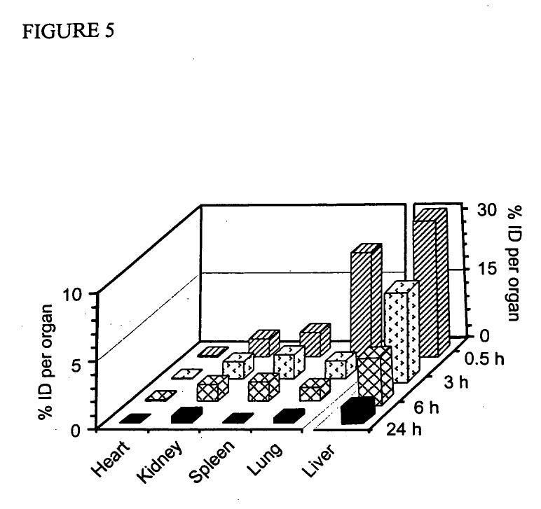

The biodistribution of ^{99m}Tc-labeled phage in normal mice is shown in FIG. 5. The lungs and liver were the organs of greatest accumulation at the earliest time with about 31% in liver, 8% in lungs and 2% in spleen at 30 minutes. Activity in all organs gradually decreased over time such that at 6 hours kidneys, spleen and lungs contained only about 1% of the injected dose, and by 24 hours liver activity was reduced to 5% and lungs to 0.39%. Values for blood were 2.5% at 30 minutes and decreased to 0.2% at 24 hours. That the lung levels rapidly decreased with time suggests that localization in this organ was not simply due to capillary trapping of a radiolabeled particle.

Infection and Inflammation in a Mouse Model

Normal mice were injected subcutaneously with each of the three live bacteria (infection-inflammation models) or the sterilized cell-free broth of each bacterial culture (inflammation models) containing, most likely, bacterial debris and intracellular materials such as endotoxins). Mice received a subcutaneous injection into one thigh of 0.1 ml of one of the six injectates (n=4). At 3 hours thereafter, mice received the labeled phage (10⁹ PFU, about 1.036 MBq) through a tail vein and, after an-additional 3 hours, the animals were imaged on an Elscint APEX 409M large view gamma camera (Hackensack, N.J.). After imaging, the organs of interest and blood were removed, weighed and counted in the gamma well counter.

Table 4 presents the biodistribution at 4 hours post administration of the labeled phage in mice induced 3 hours earlier with an infection-inflammation or a sterile inflammation in one thigh using one of the three bacterial preparations, either as live (infection-inflammation) or heat killed (sterile inflammation) preparation. As before, the liver is the organ of greatest accumulation of radioactivity in all cases. Radioactivity was also high in the stomach, and small and large intestines that may be due to the presence of endogenous bacteria in these organs. Activity in the infected/inflamed thigh was 2 to 2.5-fold higher than the normal thigh for each of the three bacterial preparations. The ratio of activity in inflamed thigh to normal thighs were lower, 1.5 to 1.8.

TABLE 4
Biodistribution at 4 hours post administration of the 99mTc-phage
in mice receiving live bacteria (infection-inflammation model) or
heat killed bacteria (sterile-inflammation model) 3 hours earlier.
Percent injected dose per organ. Mean (SD), Mean of N = 4.

| | | | | | | E. coli |
|---|---|---|---|---|---|---|
| | E. coli | E. coli 2537 | S. aureus | S. aureus | E. coli | 25922 |
| Tissue | 2537 Live | Heat Killed | Live | Heat Killed | 25922 Live | Heat Killed |
| Liver | 15.8 (0.33) | 15.12 (3.02) | 17.38 (1.34) | 16.45 (1.14) | 13.13 (1.43) | 13.87 (1.48) |
| Heart | 0.09 (0.0) | 0.08 (0.01) | 0.06 (0.01) | 0.05 (0.01) | 0.06 (0.00) | 0.06 (0.01) |
| Kidney | 1.31 (0.16) | 1.35 (0.06) | 1.08 (0.12) | 1.06 (0.20) | 1.12 (0.20) | 1.06 (0.06) |
| Lung | 1.77 (0.16) | 2.38 (0.79) | 0.74 (0.14) | 0.55 (0.19) | 1.30 (0.25) | 1.0 (0.26) |
| Spleen | 0.88 (0.08) | 1.35 (0.82) | 0.97 (0.18) | 0.69 (0.18) | 0.91 (0.16) | 1.06 (0.19) |
| Stomach | 12.58 (0.43) | 12.28 (2.84) | 15.16 (1.25) | 9.17 (2.79) | 12.20 (4.06) | 14.65 (2.65) |
| Sm. Intest. | 5.29 (0.38) | 6.85 (2.65) | 7.33 (1.31) | 5.46 (1.25) | 4.97 (0.99) | 3.61 (1.04) |
| Lrg. Intest. | 8.02 (1.96) | 6.46 (1.66) | 6.79 (0.97) | 12.65 (3.27) | 10.05 (0.84) | 11.85 (2.19) |
| Blood* | 1.38 (0.08) | 1.25 (0.14) | 1.0 (0.18) | 0.82 (0.25) | 1.06 (0.23) | 1.05 (0.17) |
| Target Thigh | 0.69 (0.1) | 0.5 (0.05) | 0.76 (0.07) | 0.40 (0.04) | 0.79 (0.07) | 0.52 (0.01) |
| Normal Thigh | 0.33 (0.04) | 0.33 (0.04) | 0.36 (0.02) | 0.27 (0.04) | 0.30 (0.05) | 0.29 (0.03) |

Blood* = per ml

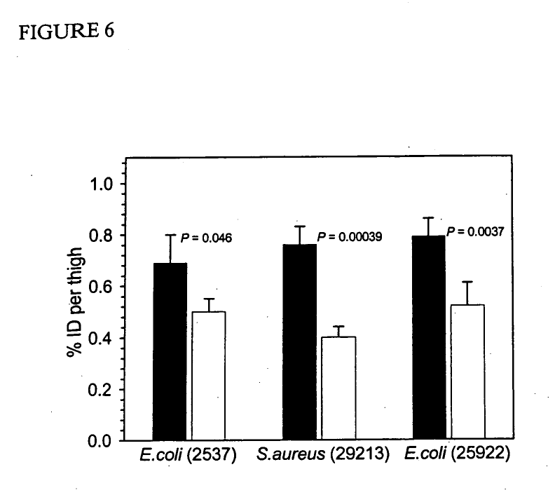

The comparison between infection-inflammation and inflammation is shown in the bar graph of FIG. 6. The difference in the percent injected dose accumulated in the infected thigh versus inflamed thigh was significant in each case (Student's t-test) at 0.69 vs 0.50 (E. coli 2537, P=0.046), 0.76 vs 0.40 (S. aureus, P=0.00039), and 0.79 vs 0.52 (E. coli 25922, P=0.0037).

In one study the live bacteria and heat killed preparation were introduced only 20 minutes before administration of the ^{99m}Tc-phage in an attempt to minimize the contribution from inflammation in the infected thigh. After 3 hours the accumulated activity in the infected thigh was 2.3-fold higher than the normal thigh, while the inflamed thigh to normal thigh ratios was 1.6. These values are essentially identical to that reported above using a 3 hours period between induction and phage administration. The percent injected dose in the normal thigh was again statistically identical (P=0.11) for both sets of animals.

### Example 6

### Imaging Infection in a Mouse

To establish that labeled phage can be used to distinguish between infection and inflammation, normal mice were injected subcutaneously with each of the three live bacteria (infection-inflammation models) or the sterilized cell-free broth of each bacterial culture (inflammation models) containing, most likely, bacterial debris and intracellular materials such as endotoxins). Mice received a subcutaneous injection into one thigh of 0.1 ml of one of the six injectates (n=4). At 3 hours thereafter, mice received the labeled phage (10⁹ PFU, about 1.036 MBq) through a tail vein and, after an additional 3 hours, the animals were imaged on an Elscint APEX 409M large view gamma camera (Hackensack, N.J.). The study was also repeated with live and a heat killed preparation with only 20 minutes instead of 3 hours between preparation of the model and administration of the labeled phage. The shorter period was selected with the purpose of minimizing the contribution from inflammation in the infection-inflammation model. After imaging, the organs of interest and blood were removed, weighed and counted in the gamma well counter.

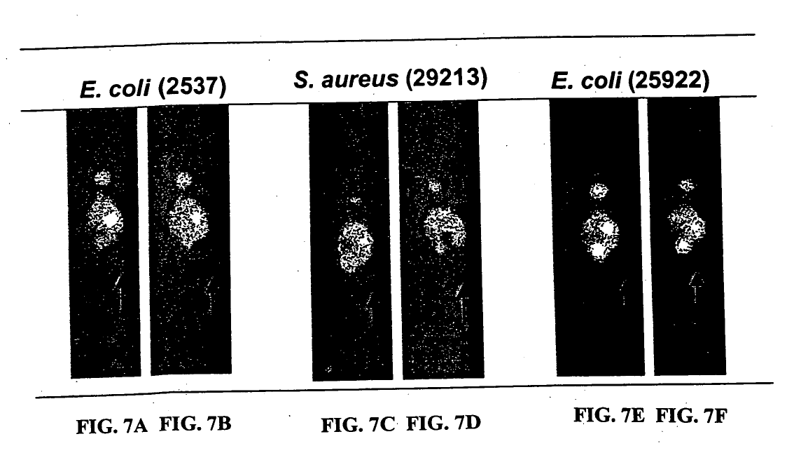

FIGS. 7A-7F present whole body images taken at 3 hours following administration of ^{99m}Tc-labeled phage to mice with the three indicated bacterial preparations as either an infection-inflammation (FIGS. 7A, 7C, and 7E) or a sterile inflammation (FIGS. 7B, 7D, and 7F) in the right thigh (indicated by arrow). The area of highest accumulation is in the abdomen, most likely liver and gut. The focal uptake in the neck is thought to be due to the small percentage of pertechnetate in the injectates localizing in the thyroid. The greater accumulation of activity in the infected thigh in comparison to the inflamed thigh is evident.

### Example 7

### Investigation of Four ^{99m}Tc Labeled Bacteriophages

The phages VD-13, E79, P22, and 60 were obtained commercially, cultured, radiolabeled with ^{99m}Tc via MAG₃ as described above, and incubated with hosts Enterococcus faecium, Pseudomonas aeruginosa, Salmonella typhimurium, and Klebsiella pneumoniae, respectively. For in vitro evaluation, 10⁸-10⁹ PFU of each ^{99m}Tc-phage was added to 10⁸ host bacteria and at least two non-host bacteria in the presence or absence of the detergent Tween®-20 and, after 15 minutes on ice, the bacterial pellets were collected, washed, and counted.

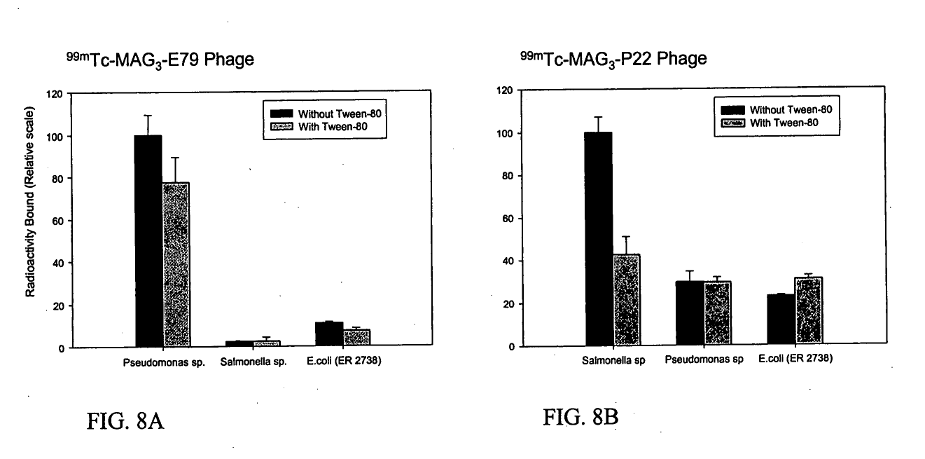

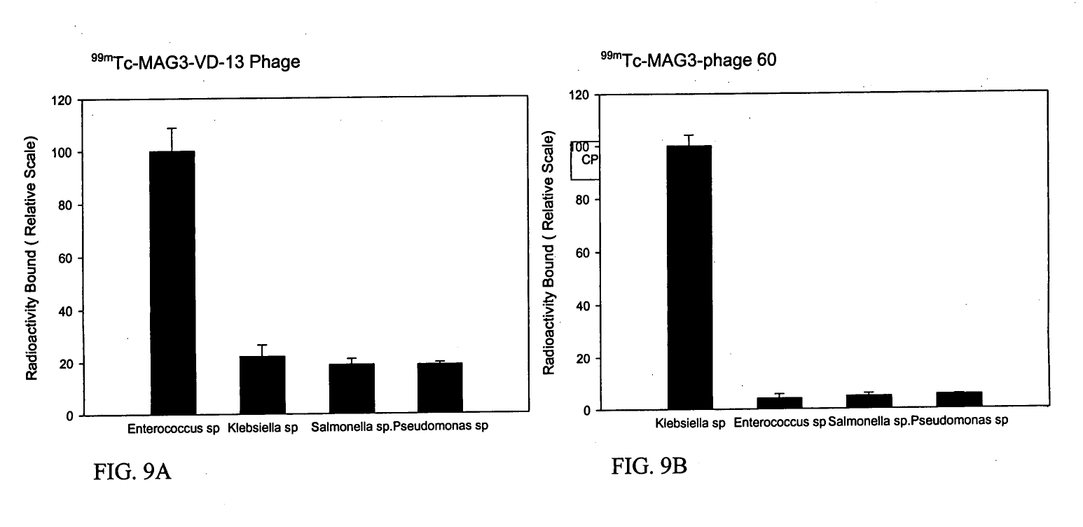

Radiochemical purity was typically greater than 90%. Each phage bound in vitro to its host at least 3-fold higher than to non-host bacteria (FIGS. 8A-8B and 9A-9B). For example, ^{99m}Tc-E79 showed 10-fold greater binding to host vs. E. coli, and 20-fold greater binding to host vs. S. typhimurium (FIG. 8A), whereas ^{99m}Tc-60 showed 20-fold greater binding to its host vs. three non-hosts (FIG. 9B). ^{99m}Tc-P22 showed 3-fold greater binding to its host vs. two non-hosts (FIG. 8B), whereas ^{99m}Tc-VD13 showed 5-fold greater binding to its host vs. three non-hosts (FIG. 9A).

Phage P22 was also conjugated to Cy™5.5 and its binding to Enterococcus faecalis, P. aeruginosa, K. pneumoniae, and Salmonella choleraesuis was investigated as described above, except that fluorescence was measured in the bacterial pellets. Only the host bacterium S. choeraesuis showed signficant retention of the Cy™5.5-conjugated phage.

In vivo studies involved injection of phage to animals infected with host or non-host bacteria. Mice received 10⁷ bacteria in one thigh, and ^{99m}Tc-phages were administered intravenously 3-5 hours later. After a further 3-4 hours, the infected and contralateral thighs were removed along with organs of interest and counted for radioactivity.

In mice, liver accumulation was high at 45% and 26% for E79 and P22 compared to 9.4% and 14.4% for 60 and VD-13, respectively, irrespective of bacteria. The infected to normal thigh ratio for ^{99m}Tc-E79 in animals infected with its host was 13.8 vs. 5.1, 6.7, and 11.8 for non-host phage 60, VD-13 and P22, respectively. Whole infected thigh accumulation for ^{99m}Tc-P22 in animals infected with its host was 1.3% ID vs. 0.72%, 0.42%, and 0.86% ID for nonspecific phage 60, VD-13 and E79, respectively. Infected thigh accumulations of VD-13 in its host infected mice were similar to that in mice infected with non-host K. pneumoniae and S. typhimurium (0.44%, 0.30% and 0.42%, respectively) and higher in non-host P. aeruginosa (1.3% ID). Similarly, ^{99m}Tc-phage 60 showed higher accumulation in P. aeruginosa infected thigh than its host K. pneumoniae (1.9% vs. 0.6% ID).

Taken together, these results demonstrate that ^{99m}Tc-labeled bacteriophage can detect and distinguish among specific host bacteria both in vitro and in vivo. Specific host binding was observed in vitro for each of the four ^{99m}Tc-phages. The in vivo studies showed similar specificity for two of the four ^{99m}Tc-phages.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

TABLE 1

| | |
|---|---|
| Bacterial Host Genus | Bacteriophage(s) Infectious for Bacterial Host |

INOVIRIDAE, genus Inovirus
long filaments with helical symmetry, circular ssDNA

| | |
|---|---|
| Clostridium | CAK1. |
| Enterobacteria | AE2, Ec9, C-2, fd, f1, (syn = f-1), HR, If1, If2, IKe, I₂-2, M13, |
| | (syn = M-13), PR64FS, SF, tf-1, X, X-2, ZG/2, ZJ2, δA. |
| Propionibacterium | NN-Propionibacterium (1). |
| Pseudomonas | Pf1, (syn = Pf-1), Pf2, Pf3. |
| Thermus | H75. |
| Vibrio | CTXΦ, fs, (syn = fs1), fs2, lvpf5, Vf12, Vf33, VPIΦ, VSK, v6, |
| | 493. |
| Xanthomonas | Cf, Cflt, Xf, (syn = xf), Xf2, φLf, φXo, φXv. |

INOVIRIDAE, genus Plectrovirus
short rods with helical symmetry, circular ssDNA

| | |
|---|---|
| Acholeplasma | MV-L1, MVL51, MVL52, MV-L59, MV-L60, 03cl, 011clr, 10tur, |
| | 143tur, 179tur, 182tur, 1304clr. |
| Mycoplasma | MV-G51, NN-Mycoplasma (1). |
| Spiroplama | SV-C1, (syn = SV1/KC3·VC3). |

LEVIVIRIDAE
quasi-icosahedral capsid, one molecule of linear ssRNA

| | |
|---|---|
| Acinetobacter | 142, 205. |
| Caulobacter | φCb5, φCb8r, φCb12r, φCb23r, φCp2, φCp14, φCr14, φCr28. |
| Enterobacteria | B6, B7, C-1, C2, FH5, Fₒlac, fr, f2, (syn = f₂), Hgal, Iα, M, |
| | MS2, M12, (syn = M-12), pilHα, Qβ, R17, (syn = R-17), SR, t, |
| | ZG/1, ZIK/1, ZJ/1, ZL/3, ZS/3, α15, μ2, (syn = μ₂). |
| Pseudomonas | PP7, PRR1, 7s, NN-Pseudomonas (1). |

LIPOTHRIXVIRIDAE
enveloped filaments, lipids, linear dsDNA

| | |
|---|---|
| Acidianus | DAFV. |
| Sulfolobus | SIFV. |
| Thermoproteus | TTV1, TTV2, TTV3, TTV4. |

MICROVIRIDAE
icosahedral capsid, circular ssDNA

| | |
|---|---|
| Bdellovibrio | MAC-1, MAC-1′, MAC-2, MAC-4, MAC-4′, MAC-5, MAC-7. |
| Chlamydia | Chp1. |
| Enterobacteria | BE/1, dφ3, dφ4, dφ5, G4, G6, G13, G14, lφ1, lφ3, lφ7, lφ9, |
| | M20, St-1, (syn = St/1), (syn = ST-1), S13, (syn = S-13), U3, WA/1, |
| | WF/1, WW/1, ZD13, α3, α10, δ1, η8, o6, φA, φR, φX174, |
| | (syn = φX), (syn = φX-174), (syn = ΦX174, ζ3. |
| Spiroplasma | SpV4. |

MYOVIRIDAE, morphotype A1
tail contractile, head isometric

| | |
|---|---|
| Acetobacter | Acm1, (syn = MOI), Acm2, Acm5, Acm6, Acm7, pAg-1, pKA-1, |
| | pKG-1, pKG-2, pKG-3, pKG-4, pOA-1. |
| Achromobacter | NN-Achromobacter (13). |
| Acinetobacter | A1, A3/2, A4, A9, A10/45, BS46, E1, E2, E7, E14, G4, HP2, |
| | HP3, HP4, 20, 59, 73, 103, 104, 108, 138, 141, 143, 196, 204, |
| | 206. |
| Actinobacillus | Aaφ23, Aaφ76, Aaφ97, Aaφ99, Aaφ247, PAA24, PAA84, φAa17, |
| | NN-Actinobacillus (1). |
| Actinoplanes | NN-Actinoplanes (1). |
| Aeromonas | Aeh2, N, PM1, TP446, 3, 4, 11, 13, 29, 31, 32, 37, 43, 43-10T, |
| | 51, 54, 55R.1, 56, 56RR2, 57, 58, 59.1, 60, 63. |
| Agrobacterium | GS2, GS6, PIIBNV6, P0362 (defective). |
| Alcaligenes | A6, A11/A79, H20. |
| Alicyclobacillus | P10. |
| Anabaena | AN-10, AN-15, A-1(L), (syn = A-1), A-2, NN-Anabaena (1). |
| Anacystis | AS-1, AS-1M, NN-Anacystis (1). |
| Aneurinobacillus | φBa1. |
| Archaebacteria | Halobacterium, Methanobacterium, Natronobacterium. |
| Azotobacter | A-11, A-14, (syn = A14), PCan, PR10, P6, P12, P14, P18, P27, |
| | P32, P38, P63. |
| Bacillus | ale1, AR1, AR2, AR3, AR7, AR9, Bace-11, (syn = 11), Bastille, |
| | BL1, BL2, BL3, BL4, BL5, BL6, BL8, BL9, BP124, BS28, |
| | BS80, Ch, CP-51, CP-54, D-5, dar1, den1, DP-7, ent2, FoS₁, |
| | FoS₂, FS₄, FS₆, FS₇, G, gal1, gamma, GE1, GF-2, GS₁, GT-1, |
| | GT-2, GT-3, GT-4, GT-5, GT-6, GT-7, GV-6, g15, I9, I10, IS₁, |
| | K, MP9, MP13, MP21, MP23, MP24, MP28, MP29, MP30, |
| | MP32, MP34, MP36, MP37, MP39, MP40, MP41, MP43, MP44, |
| | MP45, MP47, MP50, NLP-1, No.1, N17, N19, PBS1, PK1, |
| | PMB1, PMB12, PMJ1, S, SPO1, SP3, SP5, SP6, SP7, SP8, SP9, |
| | SP10, SP-15, SP50, (syn = SP-50), SP82, SST, sub1, SW, Tg8, |
| | Tg12, Tg13, Tg14, thu1, thu4, thu5, Tin4, Tin23, TP-13, TP33, |
| | TP50, TSP-1, type V, type VI, V, Vx, β22, φe, φNR2, φ25, φ63, |
| | 1, 1, 2, 2C, 3NT, 4, 5, 6, 7, 8, 9, 10, 12, 12, 17, 18, 19, 21, |
| | 138, III. |
| | The following are defective: DLP10716, DLP-11946, DPB5, |
| | DPB12, DPB21, DPB22, DPB23, GA-2, M, No.1M, PBLB, |
| | PBSH, PBSV, PBSW, PBSX, PBSY, PBSZ, phi, SPa, type 1, m |
| Bacteroides | Bf42, Bf71, NN-Bdellovibrio (1). |
| Bdellovibrio | HDC-2, MAC-6, VL-1. |
| Borrelia | NN-Borrelia (2). |
| Brochothrix | A6, A8, A9, A19, A20. |
| Burkholderia | CP75, NN-Burkholderia (1). |
| Campylobacter | C type, NTCC12669, NTCC12670, NTCC12671, NTCC12672, |
| | NTCC12673, NTCC12674, NTCC12675, NTCC12676, |
| | NTCC12677, NTCC12678, NTCC12679, NTCC12680, |
| | NTCC12681, NTCC12682, NTCC12683, NTCC12684, 32f, 111c, |
| | 191, NN-Campylobacter (2). |
| Carnobacterium | cd1. |
| Caryophanon | Cslₓ₁₃b. |
| Caulobacter | φCr24, φCr26, φCr30, φCr35. |
| Citrobacter | FC3-9. |
| Clostridium | CA5, Ca7, CEβ, (syn = 1C), CEγ, Cld1, c-n71, c-203 Tox-, DEβ, |
| | (syn = 1D), (syn = 1D^{tox+}), HM3, KM1, KT, Ms, NA1, (syn = Na1^{tox+}), |
| | PA1350e, Pfo, PL73, PL78, PL81, P1, P50, P5771, |
| | P19402, 1C^{tox+}, 2C^{tox-}, 2D, (syn = 2D^{tox+}), 3C, (syn = 3C^{tox+}), 4C, |
| | (syn = 4C^{tox+}), 56, III-1, NN-Clostridium (61). |
| Corynebacterium | CGK1 (defective). |
| Cyanobacteria | Anabaena, Anacystis, Nostoc, Plectonema, Synechococcus. |
| Cytophaga | C2, φCj1, φCj2, φCj5, φCj7, φCj8, φCj9, φCj10, φCj11, φCj12, |
| | φCj13, φCj14, φCj15, φCj16, φCj20, φCj23, φCj24, φCj25, φCj26, |
| | φCj27, φCj28, φCj29, φCj30, φCj31, φCj32, φCj33, φCj34, |
| | φCj35, φCj36. |
| Desulfovibrio | NN-Desulfovibrio (1). |
| Enterobacter | WS-EP57, 379/319. |
| Enterococcus | DF₇₈, F1, F2, 1, 2, 4, 14, 41, 867. |
| Erwinia | E15P, PEa7, Y46/(CE2). |
| Escherichia | BW73, B278, D6, D108, E, E1, E24, E41, FI-2, FI-4, FI-5, |
| | HI8A, HI8B, i, MM, Mu, (syn = mu), (syn = Mu1), (syn = Mu−1), |
| | (syn = MU−1), (syn = MuI), (syn = μ), O25, PhI-5, Pk, PSP3, P1, |
| | P1D, P2, P4 (defective), S1, Wφ), φK13, φR73 (defective), φ1, φ2, |
| | φ7, φ92, ψ (defective), 7A, 8φ, 9φ, 15 (defective), 18, 28−1, |
| | 186, 299, NN-Escherichia (2). |
| Flavobacterium | T-φD1B. |
| Fusobacterium | NN-Fusobacterium (2). |
| Gluconobacter | A-1, Gs1, Gs2, Gs3, GW6210. |
| Haemophilus | HP1, S2. |
| Halobacterium | HF1, HF2, Hs1, Ja-1, φH. |
| Hyphomicrobium | Hyfa-1, Hyfa-2, Hyfa-3, Hyfa-4. |
| Klebsiella | AIO-2, Kl₄B, Kl₆B, Kl₉, (syn = Kl9), Kl₁₄, Kl₁₅, Kl₂₁, Kl₂₈, Kl₂₉, |
| | Kl₃₂, Kl₃₃, Kl₃₅, Kl₁₀₆B, Kl₁₇₁B, Kl₁₈₁B, Kl₈₃₂B. |
| Lactobacillus | ATCC 25180, b2, FE5-B2, FE5-B3, FE5-B4, fri, FYc, hb, hv, |
| | hw, hw1, LB2, LB7, L112, (syn = 112), NCDO 01244, NHc, NTc, |
| | TKc, TMc, TZc, ΦCh38, ΦLP65, Φ1, Φ2, Φ3, Φ4, Φ5, Φ6, |
| | Φ8, Φ9, Φ204, Φ218, 032, 034, 035, 065, 0240, 0241, 0243, |
| | 0244, 0303, 0465, 0762, 01117, 206, 222a, 223-B2, 223-B3, 300, |
| | 315, 328-B1, 356, 514, 832-B1, 834-B3, 835-B11, 1097-B12, |
| | 1097-B14. |
| Lactococcus | c10III, RZh, NN-Lactococcus (1). |
| Leptospira | LE1, LE3, LE4, NN-Leptospira (1). |
| Levinea | DM-11, DM-41. |
| Listeria | A511, O1761, 4211, 4286, (syn = BO54). |
| Methanobacterium | ΦF1. |
| Methylomonas | MP3. |
| Mycobacterium | I3. |
| Mycoplasma | MVBr1. |
| Myxococcus | MX-1, MX4, MX41, MX43, Mv-1g1, Mv-1g2, φa, φb, φm, φv, |
| | φ2. |
| Natronobacterium | ΦCh1. |
| Neisseria | Group I, group II, NP1. |
| Nostoc | N−1. |
| Paenibacillus | BL2, BP123, BP124, BP128, EP1, EPy-1, EPy-2, EPy-3, EPy-4, |
| | EPy-5, FoP1, FP1, FP2, FP3, FP4, FP5, FP6, FP7, FP10, FP11, |
| | GP1, IP1, PBL0.5c, SP1, SPy-1, SPy-2, SPy-3. |
| Pasteurella | AU, VL, TX, φPhA1, 1, 2, 10, 3/10, 4/10, 115/10, 895. |
| Plectonema | NN-Plectonema (1). |
| Proteus | Pm5, 13vir, 2/44, 4/545, 6/1004, 13/807, 20/826, 57, 67b, 78, |
| | 107/69, 121. |
| Pseudoalteromonas | H7/2, H71/1, H71/5, H106-1, H114/2, 6-8a, 6-42c, 6-62c, 12-13a, |
| | 12-13b, 12-41b. |
| Pseudomonas | AI-1, AI-2, B17, B89, CB3, Col 2, Col 11, Col 18, Col 21, C154, |
| | C163, C167, C2121, E79, F8, ga, gb, H22, K₁, M4, N₂, Nu, |
| | PB-1, (syn = PB1), pf16, PMN17, PP1, PP8, Psa1, PsP1, PsP2, |
| | PsP3, PsP4, PsP5, PS3, PS17, PTB80, PX4, PX7, PYO1, PYO2, |
| | PYO5, PYO6, PYO9, PYO10, PYO13, PYO14, PYO16, PYO18, |
| | PYO19, PYO20, PYO29, PYO32, PYO33, PYO35, PYO36, |
| | PYO37, PYO38, PYO39, PYO41, PYO42, PYO45, PYO47, |
| | PYO48, PYO64, PYO69, PYO103, P1K, SLP1, SL2, S₂, UNL-1, |
| | wy, Ya₁, Ya₄, Ya₁₁, φBE, φCTX, φC17, φKZ, (syn = φKZ), φ-LT, |
| | Φmu78, φNZ, φPLS-1, φST-1, φW-14, φ-2, 1/72, 2/79, 3, 3/DO, |
| | 4/237, 5/406, 6C, 6/6660, 7, 7v, 7/184, 8/280, 9/95, 10/502, |
| | 11/DE, 12/100, 12S, 16, 21, 24, 25F, 27, 31, 44, 68, 71, 95, |
| | 109, 188, 337, 352, 1214, NN-Pseudomonas (23). |
| Rhizobia | a, c, CM1, (syn = CM₁), CM2, (syn = CM₂), CM3, CM4, CM5, |
| | CM6, CM7, CM8, CM9, CM20, CM21, CT1, CT3, CT4, CT5, |
| | CT6, E, e, FIA, I, J, j, l, m, RL4, TN1, WT1, φgal1/OW, |
| | φgal1/R, φgal3/OW, φgal3/R, φM12, φ18, φ2193/2, 4, 7-7-1, 16- |
| | 7-1, 16-35-5. |
| Rhodobacter | φRsD, φRsV. |
| Salinivibrio | G3, UTAK. |
| Salmonella | b, Beccles, CT, d, Dundee, f, Fels 2, GI, GIII, GVI, GVIII, k, |
| | K, i, j, L, O1, (syn = O-1), (syn = O₁), (syn = O-I), (syn = 7), O2, |
| | O3, P3, P9a, P10, Sab3, Sab5, San15, San17, SI, Taunton, ViI, |
| | (syn = Vi1), 9, NN-Salmonella (1). |
| Saprospira | NN-Saprospira (1). |
| Serpulina | VSH-1, NN-Serpulina (1). |
| Serratia | A2P, PS20, SMB3, SMP, SMP5, SM2, V40, V56, κ, φCP-3, |
| | φCP-6, 3M, 10/1a, 20A, 34CC, 34H, 38T, 345G, 345P, 501B. |
| Shigella | Fsa, (syn = a), FS_{D2d}, (syn = D2d), (syn = W₂d), FS_{D2E}, (syn = W₂e), |
| | fv, F6, f7.8, H-Sh, PE5, P90, SfII Sh, SH_{III}, SH_{IV}, (syn = HIV), |
| | SH_{VI}, (syn = HVI), SHV_{VIII}, (syn = HVIII), SKγ66, (syn = gamma |
| | 66), (syn = γ66), (syn = γ66b), SK_{III}, (syn = SIIIb), (syn = III), SK_{IV}, |
| | (syn = S_{IVa}), (syn = IV), SK_{IVa}, (syn = S_{IVAa}), (syn = IVA), SK_{VI}, |
| | (syn = KVI), (syn = S_{VI}), (syn = VI), SK_{VIII}, (syn = S_{VIII}), (syn = VIII), |
| | SK_{VIIIA}, (syn = S_{VIII}A), (syn = VIIIA), ST_{VI}, ST_{IX}, ST_{XI}, ST_{XII}, |
| | S66, W₂, (syn = D2c), (syn = D20), φI, φIV₁, 3-SO-R, 8368-SO-R. |
| Sphingomonas | PAU. |
| Staphylococcus | A, EW, K, Ph5, Ph9, Ph10, Ph13, P1, P2, P3, P4, P8, P9, P10, |
| | RG, S_{B-1}, (syn = Sb-1), S3K, Twort, φSK311, φ812, 06, 40, 58, |
| | 119, 130, 131, 200, 1623. |
| Streptococcus | EJ-1, NN-Streptococcus (1). |
| Streptomyces | SK1, type IV. |
| Synechococcus | S-BM1, S-BS1, S-PM1, S-PS1, S-PWM, S-PWM1, S-PWM2, S- |
| | PMW4, S-WHM1, S-3(L), S-6(L), (syn = S-6L), S-7(L), (syn = S- |
| | 7L), NN-Synechococcus (1). |
| Tetragenococcus | φ7116. |
| Thermus | φYS40. |
| Thiobacillus | HT-2. |
| Vibrio | CP-T1, ET25, kappa, K139, LaboI, OXN-69P, OXN-86, O6N- |
| | 21P, PB-1, P147, rp-1, SE3, VA-1, (syn = VcA-1), VcA-2, VcA- |
| | 3, VP1, VP2, VP4, VP7, VP8, VP9, VP10, VP17, VP18, VP19, |
| | X29, (syn = 29 d'Herelle), β, ΦHAWI-1, ΦHAWI-2, ΦHAWI-3, |
| | ΦHAWI-4, ΦHAWI-5, ΦHAWI-6, ΦHAWI-7, ΦHAWI-8, |
| | ΦHAWI-9, ΦHAWI-10, ΦHC1-1, ΦHC1-2, ΦHC1-3, ΦHC1-4, |
| | ΦHC2-1, ΦHC2-2, ΦHC2-3, ΦHC2-4, ΦHC3-1, ΦHC3-2, |
| | ΦHC3-3, ΦHD1S-1, ΦHD1S-2, ΦHD2S-1, ΦHD2S-2, ΦHD2S-3, |
| | ΦHD2S-4, ΦHD2S-5, ΦHDO-1, ΦHDO-2, ΦHDO-3, ΦHDO-4, |
| | ΦHDO-5, ΦHDO-6, ΦKL-33, ΦKL-34, ΦKL-35, ΦKL-36, |
| | ΦKWH-2, ΦKWH-3, ΦKWH-4, ΦMARQ-1, ΦMARQ-2, |
| | ΦMARQ-3, ΦMOAT-1, ΦO139, ΦPEL1A-1, ΦPEL1A-2, |
| | ΦPEL8A-1, ΦPEL8A-2, ΦPEL8A-3, ΦPEL8C-1, ΦPEL8C-2, |
| | ΦPEL13A-1, ΦPEL13B-1, ΦPEL13B-2, ΦPEL13B-3, ΦPEL13B- |
| | 4, ΦPEL13B-5, ΦPEL13B-6, ΦPEL13B-7, ΦPEL13B-8, |
| | ΦPEL13B-9, ΦPEL13B-10, φVP143, φVP253, Φ16, φ138, 1-11, |
| | 5, 13, 14, 16, 24, 32, 493, 6214, 7050, 7227, II, (syn = group II), |
| | (syn = φ2), V, VIII, NN-Vibrio (13). |
| Xanthomonas | HP1, OX1, (syn = XO1), OX2, SBX-1, XCVP₁, XP5, XTP1. |
| Yersinia | H, H-1, H-2, H-3, H-4, Lucas 110, Lucas 303, Lucas 404, YerA3, |
| | YerA7, YerA20, YerA41, 3/M64-76, 5/G394-76, 6/C753-76, |
| | 8/C239-76, 9/F18167, 1701, 1710. |

MYOVIRIDAE, morphotype A2
tail contractile, head elongated (length/width ratio = 1.3-1.8)

| | |
|---|---|
| Acinetobacter | E4, E5, HP1, 102, 106, 133. |
| Aeromonas | Aeh1, F, PM2, 1, 25, 31, 40RR2.8t, (syn = 44R), (syn = 44RR_{2.8}t), |
| | 65. |
| Bdellovibrio | MAC-3. |
| Burkholderia | 42. |
| Citrobacter | FC3-1, FC3-2, FC3-3, FC3-4, FC3-6, FC3-7. |
| Clostridium | NB1^{tox+}, α1. |
| Enterobacter | WS-EP32, WS-EP94, WS-EP96, 1, (syn = PsaeI), X. |
| Enterobacteria | Citrobacter, Enterobacter, Erwinia, Escherichia, Klebsiella, |
| | Morganella, Proteus, Providencia, Salmonella, Serratia, Shigella, |
| | Yersinia. |
| Escherichia | AB48, CM, C4, C16, DD-VI, (syn = D_{d}-Vi), (syn = DDVI), (syn = DDVi), |
| | E4, E7, E28, FI1, FI3, H, H1, H3, H8, K3, M, N, ND- |
| | 2, ND-3, ND4, ND-5, ND6, ND-7, Ox-1, (syn = OX1), (syn = 11F), |
| | Ox-2, (syn = Ox2), (syn = OX2), Ox-3, Ox-4, Ox-5, (syn = OX5), |
| | Ox-6, (syn = 66F), (syn = φ66t), (syn = φ66t-), O111, PhI-1, |
| | RB42, RB43, RB49, RB69, S, Sal-1, Sal-2, Sal-3, Sal-4, Sal-5, |
| | Sal-6, TC23, TC45, TuII*-6, (syn = TuII*), TuII*-24, TuII*46, |
| | TuII*-60, T2, (syn = gamma), (syn = γ), (syn = PC), (syn = P.C.), |
| | (syn = T-2), (syn = T₂), (syn = P₄), T4, (syn = T-4), (syn = T₄), T6, |
| | T35, α1, 1, 1A, 3, (syn = Ac3), 3A, 3T⁺, (syn = 3), (syn = M1), |
| | 5Φ, (syn = Φ5), 9266Q. |
| Klebsiella | AIO-1, AO-1, AO-2, AO-3, FC3-10, K, kl₁, (syn = Kl1), Kl₂, |
| | (syn = Kl2), Kl₃, (syn = Kl3), (syn = K170/11), Kl₄, (syn = Kl4), Kl₅, |
| | (syn = Kl5), Kl₆, (syn = Kl6), Kl₇, (syn = Kl7), Kl₈, (syn = Kl8), |
| | Kl₁₉, (syn = Kl9), Kl₂₇, (syn = Kl27), Kl₃₁, (syn = Kl31), Kl₃₅, |
| | Kl₁₇₁B, II, VI, IX. |
| Levinea | DM-51. |
| Morganella | 50, 5845. |
| Proteus | 9/0, 22/608, 30/680. |
| Providencia | 8893, 9266. |
| Rhizobia | NN-Rhizobium (1). |
| Rhodobacter | I-2. |
| Saccharomonospora | 108/106. |
| Salmonella | N-1, N-5, N-10, N-17, N-22 |
| Serratia | SMB2, SMP2. |
| Shigella | F7, (syn = FS7), (syn = K29), F10, (syn = FS10), (syn = K31), I₁, |
| | (syn = alfa), (syn = FSα), (syn = K18), (syn = α), I₂, (syn = a), (syn = K19), |
| | SG₃₅, (syn = G35), (syn = SO-35/G), SG₅₅, (syn = SO-55/G), |
| | SG₃₂₀₁, (syn = SO-3201/G), SH_{II}, (syn = HII), SH_{V}, (syn = SHV), |
| | SH_{X}, SHX, SK_{II}, (syn = K2), (syn = KII), (syn = S_{II}), (syn = SsII), |
| | (syn = II), SK_{IV}, (syn = S_{IVb}), (syn = SsIV), (syn = IV), SK_{IVa}, (syn = S_{IVab}), |
| | (syn = SsIVa), (syn = IVa), SK_{V}, (syn = K4), (syn = KV), |
| | (syn = SV), (syn = SsV), (syn = V), SK_{X}, (syn = K9), (syn = KX), |
| | (syn = SX), (syn = SsX), (syn = X), ST_{V}, (syn = T35), (syn = 35-50- |
| | R), ST_{VIII}, (syn = T8345), (syn = 8345-SO-S-R), W₁, (syn = D8), |
| | (syn = FS_{D8}), W₂a, (syn = D2A), (syn = FS₂ₐ). |
| Vibrio | KVP20, KVP40, nt-1, O6N-22P, P68. |
| Yersinia | PST, 1/F2852-76. |

MYOVIRIDAE, morphotype A3
tail contractile, head elongated (length/width ratio = 2 or more)

| | |
|---|---|
| Enterobacteria | Erwinia, Salmonella. |
| Erwinia | E16B.] |
| Salmonella | 11, 12, 16-19, 20.2, 36, 449C/C178, 966A/C259. |
| Spirochaeta? | NN-Spirochaeta? (1). |

PLASMAVIRIDAE
pleomorphic, envelope, lipids, no capsid, circular supercoiled dsDNA

| | |
|---|---|
| Acholeplasma | L2, (syn = MVL2), (syn = MV-L2), L172, (syn = MV-L172), MV-Lg- |
| | L172, (syn = MV-Lg-pS2-L172), M1, (syn = MV-M1), O-1, |
| | (syn = MV-O1), 1302. |

PODOVIRIDAE, morphotype C1
tail short and noncontractile, head isometric

| | |
|---|---|
| Acholeplasma | BN1, MV-L3, (syn = MVL3). |
| Achromobacter | OXN-36P, NN-Achromobacter (1). |
| Acinetobacter | A31, A33, A34, A36, A37, BP1, B₉GP, P78, 56. |
| Actinomyces | Av-1, Av-2, Av-3, BF307, CT1, CT2, CT3, CT6, CT7, 1281. |
| Aeromonas | AA-1. |
| Agrobacterium | PR-590a, PR-1001, PsR-1012, PS-192, PIIBNV6-C. |
| Alcaligenes | Z-1/H-16. |
| Anabaena | AC-1. |
| Anacystis | AN-20, AN-22, AN-24, A-1, A-4(L). |
| Aneurinobacillus | ΦBA1. |
| Arthrobacter | AN25S1, AN29R2. |
| Azotobacter | A-12, (syn = A12), A-21, (syn = A21), A-22, A-23, A-24, A41. |
| Bacillus | 4 (B. megaterium), 4 (B. sphaericus). |
| Bacteroides | Bf-41. |
| Bartonella | NN-Bartonella (1). |
| Brucella | A422, Bk, (syn = Berkeley), BM₂₉, FO₁, (syn = FO1), (syn = FQ1), |
| | D, FP₂, (syn = FP2), (syn = FD2), Fz, (syn = Fz75/13), (syn = Firenze 75/13), |
| | (syn = Fi), F₁, (syn = F1), F₁m, (syn = Flm), (syn = Fim), |
| | F₁U, (syn = FlU), (syn = FiU), F₂, (syn = F2), F₃, (syn = F3), |
| | F₄, (syn = F4), F₅, (syn = F5), F₆, F₇, (syn = F7), F₂₅, (syn = F25), |
| | (syn = f25), F₂₅U, (syn = F₂₅u), (syn = F25U), (syn = F25V), F₄₄, |
| | (syn = F44), F₄₅, (syn = F45), F₄₈, (syn = F48), I, Im, M, MC/75, |
| | M51, (syn = M85), P, (syn = D), S708, R, Tb, (syn = TB), (syn = Tbilisi), |
| | W, (syn = Wb), (syn = Weybridge), X, 3, 6, 7, 10/1, |
| | (syn = 10), (syn = F₈), (syn = F8), 12m, 24/II, (syn = 24), (syn = F₉), |
| | (syn = F9), 45/III, (syn = 45), 75, 84, 212/XV, (syn = 212), (syn = F₁₀), |
| | (syn = F10), 371/XXIX, (syn = 371), (syn = F₁₁), (syn = F11), |
| | 513. |
| Caulobacter | φCd1, φCr40, φCr41. |
| Citrobacter | FC3-8. |
| Clostridium | CA1, HMT, HM2, PF1, P-₂₃, P-₄₆, Q-₀₅, Q-₀₆, Q-₁₆, Q-₂₁, Q-₂₆, |
| | Q-₄₀, Q-₄₆, S₁₁₁, SA₀₂, WA₀₁, WA₀₃, W₁₁₁, W₅₂₃, 80. |
| Cyanobacteria | Anabaena, Anacystis, Plectonema, Synechococcus. |
| Desulfovibrio | NN-Desulfovibrio (1). |
| Endosymbionts | APSE-1, NN-Endosymbionts (1). |
| Enterobacter | WS-EP13, WS-EP19. |
| Enterobacteria | Citrobacter, Enterobacter, Erwinia, Escherichia, Klebsiella, |
| | Morganella, Proteus, Providencia, Salmonella, Serratia, Shigella, |
| | Xenorhabdus, Yersinia. |
| Enterococcus | D1, SB24, 2BV, 182, 225. |
| Erwinia | PEal(h), S1, φM1. |
| Escherichia | CFO103, HK620, J, K, K1F, m59, no. A, no. E, no. 3, no. 9, N4, |
| | sd, (syn = Sd), (syn = S_{D}), (syn = S_{d}), (syn = s_{d}), (syn = SD), (syn = CD), |
| | T3, (syn = T-3), (syn = T₃), T7, (syn = T-7), (syn = T₇), WPK, |
| | W31, Δ^{H}, φC3888, φK3, φK7, φK12, φV-1, Φ04-CF, Φ05, Φ06, |
| | Φ07, φ1, φ1.2, φ20, φ95, φ263, φ1092, φI, φII, (syn = φW), Ω8, |
| | 1, 3, 7, 8, 26, 27, 28-2, 29, 30, 31, 32, 38, 39, 42, 933W, NN- |
| | Escherichia (1). |
| Flavobacterium | φCB38. |
| Fusobacterium | fv83-554/3, fv88-531/2, 227. |
| Gluconobacter | JW2040. |
| Hyphomicrobium | Hyza-38, Hyφ1A, Hyφ22a, Hyφ30, Hy-12, Hy71, ZV-260, ZV- |
| | 580, ZV-622, 1348, 1458. |
| Klebsiella | CI-1, Kl₄B, Kl₈, Kl₁₁, Kl₁₂, Kl₁₃, Kl₁₆, Kl₁₇, Kl₁₈, Kl₂₀, Kl₂₂, Kl₂₃, |
| | Kl₂₄, Kl₂₆, Kl₃₀, Kl₃₄, Kl₁₀₆B, Kl₁₆₅B, Kl₃₂₈B, KLXI, K328, |
| | P5046, 11, 380, III, IV, VII, VIII. |
| Kluyvera | Kvp1. |
| Lactococcus | NN-Lactococcus (1). |
| Levinea | DM-61. |
| Methylobacter | gb2t, ΦMT1. |
| Methylocystis | 63f, (syn = 63), (syn = 63-F) |
| Methylomonas | cm4, cm4-9, cm-68a, gb2-80, gb4, (syn = gb-4), gb4-9, M1, |
| | ΦMT2, ΦMT3, ΦMT4, 4N°9, 4N°8. |
| Methylophilus | ΦKISR1. |
| Micrococcus | C1. |
| Morganella | 10041/2815. |
| Mycoplasma | Hr1, P1. |
| Myxococcus | Mv8g1, Mv8g2, Mx8, Mx9, Mx81. |
| Paenibacillus | BP153. |
| Pasteurella | 3, 22, 55, 115, 896, 994, 995. |
| Plectonema | AT, GM, GIII, LPP-1, SPI, WA. |
| Proteus | Pm1, Pm3, Pm4, Pm6, Pm7, Pm9, Pm10, Pm11, Pv2, π1, φm, |
| | 7/549, 9B/2, 10A/31, 12/55, 14, 15, 16/789, 17/971, 19A/653, |
| | 23/532, 25/909, 26/219, 27/953, 32A/909, 33/971, 34/13, 65, |
| | 5006M, 7480b, VI. |
| Providencia | PL25, PL26, PL37, 9211/9295, 9213/9211b, 9248.] |
| Pseudoalteromonas | H71/2, H00/1, 10-33b, 10-94a. |
| Pseudomonas | A856, B26, CI-1, CI-2, C5, D, gh-1, F116, HF, H90, K₅, K₆, |
| | K104, K109, K166, K267, N₄, N₅, O6N-25P, PE69, Pf, PPN25, |
| | PPN35, PPN89, PPN91, PP2, PP3, PP4, PP6, PP7, PP8, PP56, |
| | PP87, PP114, PP206, PP207, PP306, PP651, Psp231a, Pssy401, |
| | Pssy9220, PS₁, PTB2, PTB20, PTB42, PX1, PX3, PX10, PX12, |
| | PX14, PYO70, PYO71, R, SH6, SH133, tf, Ya₅, Ya₇, φBS, |
| | ΦKf77, φ-MC, ΦmnF82, φPLS27, φPLS743, φS-1, 1, 2, 2, 3, 4, |
| | 5, 6, 7, 7, 8, 9, 10, 11, 12, 12B, 13, 14, 15, 14, 15, 16, 17, |
| | 18, 19, 20, 20, 21, 21, 22, 23, 23, 24, 25, 31, 53, 73, 119x, |
| | 145, 147, 170, 267, 284, 308, 525, NN-Pseudomonas (5). |
| Rhizobia | F9, LP, MM1C, MM1H, (syn = MM₁), RC2, RC3, RS2, R2V, S, |
| | SP, ST1, U-mole, φCC814/1, φCC814/2, φCC814/3, φCC814/4, |
| | φ2042, φ2193/1, φ2193/2, φ2200, φ5114, 2, 2a, 6, 16-3-2, 16-6- |
| | 14, NN-Rhizobia (1). |
| Rhodobacter | RS1. |
| Rhodopseudomonas | Rp1.Rp1, φBHG1. |
| Rickettsia | NN-Rickettsia (1). |
| Roseobacter | SIO1. |
| Saccharomonospora | 114. |
| Salmonella | a, B.A.O.R., e, G4, GIII, L, LP7, M, MG40, N-18, PSA68, P4, |
| | P9c, P22, (syn = P₂₂), (syn = PLT22), (syn = PLT₂₂), P22a1, P22-4, |
| | P22-7, P22-11, SNT-1, SNT-2, SP6, ViIII, ViIV, ViV, ViVI, |
| | ViVII, Worksop, ε₁₅, ε₃₄, 1, 37, 1(40), (syn = φ1[40]), 1, 42₂, 2, |
| | 2.5, 3b, 4, 5, 6, 14(18), 8, 14(6, 7), 10, 27, 28B, 30, 31, 32, 33, |
| | 34, 36, 37, 39, 1412. |
| Serratia | E20, P8, Sa1, SM4, η, ΦCP6-4, 5E, 34D, 38B, 224D1, 224D2, |
| | 2847/10b. |
| Shigella | DD-2, Sf6, FS₁, (syn = F1), SF₆, (syn = F6), SG₄₂, (syn = SO-42/G), |
| | SG₃₂₀₃, (syn = SO-3203/G), SK_{F12}, (syn = SsF₁₂), (syn = F₁₂), (syn = F12), |
| | ST_{II}, (syn = 1881-SO-R), γ66, (syn = gamma 66a), (syn = Ssγ66), |
| | φ2. |
| Spiroplasma | ag, ai, AV9/3, ESV, HSV, NSV, SVC3, (syn = SV-C3), (syn = C3), |
| | SVC3/SMCA, SVC3/608, WSV. |
| Staphylococcus | STC1, (syn = stc1), STC2, (syn = stc2), 44AHJD, 68. |
| Streptococcus | a, C1, F_{LO}Ths, H39. |
| Streptomyces | CRK, SLE111, Φ17, (syn = φ17), (syn = 2a), 1, 9, 14, 24. |
| Synechococcus | S-BBP1, SM-1, S-PWP1, S-5(L), (syn = S5-L). |
| Thermoactinomyces | Ta₁. |
| Veillonella | N2, N11. |
| Vibrio | e1, e2, e3, e4, e5, FK, G, J, K, nt-6, N1, N2, N3, N4, N5, |
| | O6N-34P, OXN-72P, OXN-85P, OXN-100P, P, Ph-1, PL163/10, |
| | Q, S, T, φ92, 1-9, 37, 51, 57, 70A-8, 72A-4, 72A-10, 110A-4, |
| | 333, 4996, I, (syn = group I), III, (syn = group III), VI, (syn = A- |
| | Saratov), VII, IX, X, NN-Vibrio (6). |
| Xanthomonas | RR68. |
| Xenorhabdus | XPL. |
| Yersinia | D'Herelle, EV, H, Kotljarova, PTB, R, Y, YerA41, φYerO3-12, |
| | 3, 4/C1324-76, 7/F783-76, 903 |

PODOVIRIDAE, morphotype C2
tail short and noncontractile (length/width ratio = 1.4)

| | |
|---|---|
| Bacillus | AR13, BPP-10, BS32, BS107, B1, B2, GA-1, GP-10, GV-3, GV- |
| | 5, g8, MP20, MP27, MP49, Nf, PP5, PP6, SF5, Tg18, TP-1, |
| | Versailles, φ15, φ29, 1-97, 837/IV, NN-Bacillus (1). |
| Kurthia | 6, 7. |
| Lactococcus | asccφ28, P034, NN-Lactococcus (4). |
| Streptococcus | Cp-1, Cp-5, Cp-7, Cp-9, Cp-10. |
| Vibrio | pA1, 7. |

PODOVIRIDAE, morphotype C3
tail short and noncontractile (length/width ratio = 2.5 or more)

| | |
|---|---|
| Enterococcus | C2, C2F, E3, E62. |
| Enterobacteria | Erwinia, Escherichia, Proteus, Salmonella, Yersinia. |
| Erwinia | Erh1, E16P. |
| Escherichia | Esc-7-11. |
| Lactococcus | KSY1, KSY2. |
| Levinea | DM-31. |
| Proteus | 13/3a. |
| Salmonella | SNT-3, 7-11, 40.3. |
| Vibrio | 7-8, 70A-2, 71A-6, 72A-5, 72A-8, 108A-10, 109A-6, 109A-8, |
| | 110A-1, 110A-5, 110A-7. |
| Yersinia | 1/M6176. |

SIPHOVIRIDAE, morphotype B1
tail long and noncontractile, head isometric

| | |
|---|---|
| Achromobacter | NN-Achromobacter (5). |
| Acidiphilium | ΦAc-1. |
| Acinetobacter | E6, E8, E9, E13, E15, 1, 11, 66. |
| Actinobacillus | PAA17, PAA23, NN-Actinobacillus (2). |
| Actinomadura | φAC1, φAC3. |
| Actinomyces | CT4, CT8. |
| Aeromonas | PM3, PM4, PM5, PM6. |
| Agrobacterium | La₆k₁, Lcg, Lc-58, LHIIBNV6-2, LHIIBV7-2, Lr-4, LV-1, |
| | (syn = PV-1), LIIBNV6-1, LIIBV7-1, PA6, PB2A, (syn = PB₂A), |
| | PS8, (syn = PS-8), R4, 70-1, 70-2, 70-3, 70-4, 70-5, |
| | 70-6, 70-7. Ψ, ω, (syn = PB6), (syn = PB-6), (syn = Ω). |
| Alcaligenes | A5/A6, A5/415, A20/415, A64/A62, A74/A3, A86/A88, |
| | ΦAE5, 8764. |
| Amycolatopsis | W2, W4, W7, W11. |
| Ancalomicrobium | Ev, Sp, Va. |
| Archaebacteria | Halobacterium, Methanobacterium. |
| Arthrobacter | AC23R-3, AC201-S, AGL1, AGL2, AGL3, AGL4, AGL5, |
| | AGL6, AGL8, AGL11, AGL12, AGL13, AGL16, AGL17, |
| | AN31S-1, ARA3, ARA8, ARA9, Arp, ASP2, ASP4, ASP7, |
| | ASP16, BK1, φAAU2, φAg8010. |
| Asticcacaulis | φAc11, φAc12, φAc13, φAc14, φAc15, φAc20, φAc31, |
| | φAc33, φAc35, φAc36, φAc37, φAc38, φAc39, φAc45, |
| | φAc46, φAc57, φAc59. |
| Azospirillum | Ab-1, Al-1. |
| Azotobacter | A13, A31. |
| Bacillus | A, aizl, Al-K-I, B, BCJA1, BC1, BC2, BLL1, BL1, BP142, |
| | BSL1, BSL2, BS1, BS3, BS8, BS15, BS18, BS22, BS26, |
| | BS28, BS31, BS104, BS105, BS106, BTB, B1715V1, C, CK- |
| | 1, Col1, Cor1, CP-53, CS-1, CS₁, D, D, D, D5, ent1, FP8, |
| | FP9, FS₁, FS₂, FS₃, FS₅, FS₈, FS₉, G, GH8, GT8, GV-1, |
| | GV-2, GT-4, g3, g12, g13, g14, g16, g17, g21, g23, g24, |
| | g29, H2, kenl, KK-88, Kum1, Kyu1, J7W-1, LP52, (syn = LP- |
| | 52), L₇, Mex1, MJ-1, mor2, MP-7, MP10, MP12, MP14, |
| | MP15, Neo1, N°2, N5, N6P, PBC1, PBLA, PBP1, P2, S-a, |
| | SF2, SF6, Sha1, Sil1, SPO2, (syn = ΦSPP1), SPβ, STI, ST₁, |
| | SU-11, t, Tb1, Tb2, Tb5, Tb10, Tb26, Tb51, Tb53, Tb55, |
| | Tb77, Tb97, Tb99, Tb560, Tb595, Td8, Td6, Td15, Tg1 |
| | Tg4, Tg6, Tg7, Tg9, Tg10, Tg11, Tg13, Tg15, Tg21, Tin1, |
| | Tin7, Tin8, Tin13, Tm3, Toc1, Tog1, tol1, TP-1, TP-10ᵥᵢᵣ, |
| | TP-15c, TP-16c, TP-17c, TP-19, TP35, TP51, TP-84, Tt4, |
| | Tt6, type A, type B, type C, type D, type E, Tφ3, VA-9, W, |
| | wx23, wx26, Yun1, α, γ, ρ11,, φmed-2, φT, φμ-4, φ3T, φ75, |
| | φ105, (syn = φ105), 1A, 1B, 1-97A, 1-97B, 2, 2, 3, 3, 3, 5, |
| | 12, 14, 20, 30, 35, 36, 37, 38, 41C, 51, 63, 64, 138D, I, II |
| | IV, NN-Bacillus (13). |
| Bacteroides | adl₂, Baf-44, Baf-48B, Baf-64, Bf-1, Bf-52, B40-8, F1, β1, |
| | φA1, φBr01, φBr02, 11, 67.1, 67.3, 68.1, NN-Bacteroides (3). |
| Bifidobacterium | Bir. |
| Bordetella | 134, NN-Bordetella (3). |
| Borrelia | NN-Borrelia (1). |
| Brevibacillus | 1P⁺f. |
| Brevibacterium | Ap85III, BB1, BB4, BB8, BB10, BB12, BB14, BFK20, |
| | Bf145, Bf203, Bf209, BK1, EφB, EΦ-y, HΦ, P465, P468II, |
| | φB, φGA1, φ3001, φ4002. |
| Brochothrix | A2, A3, A4, A5, A7, A10, A11, A12, A13, A14, A15, A16, |
| | A17, BL3, MT, NF5. |
| Campylobacter | Vfi-6, (syn = V19), Vfv-3, V2, V3, V8, V16, (syn = Vfi-1), |
| | V19, V20(V45), V45, (syn = V-45), NN-Campylobacter (1). |
| Caryophanon | Cslₓ₁₃a, Ctₖₐₛ. |
| Caulobacter | φCr1, φCr22, φ101, φ102, φ118, φ151. |
| Clavibacter | ClmX, (syn = CONX), C1mXC, (syn = CONXC), C1m8, (syn = CON8), |
| | (syn = CN8), (syn = φCN8), Clm11, (syn = CON11), |
| | (syn = CN11), (syn = φCN11), CMP1, NN-Clavibacter (1). |
| Clostridium | C, CA2, CA3, CPT1, CPT4, c1, c4, c5, HM7, H₁₁/A₁, |
| | H₁₈/A₁, H₂₂/S₂₃, H₁₅₈/A₁, K₂/A₁, K₂₁/S₂₃, M_{L}, NA2^{tox-}, Pf2, |
| | Pf3, Pf4, S₉/S₃, S₄₁/A₁, S₄₄/S₂₃, α2, 41, 112/S₂₃, 214/S₂₃, |
| | 233/A₁, 234/S₂₃, 235/S₂₃, II-1, II-2, II-3, NN-Clostridium (12). |
| Corynebacterium | A, A2, A3, A101, A128, A133, A137, A139, A155, A182, |
| | B, BF, B17, B18, B51, B271, B275, B276, B277, B279, |
| | B282, C, cap₁, CC1, CG1, CG2, CG33, CL31, Cog, (syn = CG5), |
| | D, E, F, H, H-1, hq₁, hq₂, I₁/H₃₃, I₁/31, J, K, K, |
| | (syn = K^{tox-}), L, L, (syn = K^{tox+}), M, MC-1, MC-2, MC- |
| | 4, MLMa, N, O, ov₁, ov₂, ov₃, P, P, R, RP6, Rₛ29, S, T, U, |
| | UB₁, ub₂, UH₁, UH₃, uh₃, uh₅, uh₆, β, (syn = β^{tox+}), β^{hv64}, |
| | βvir, γ, (syn = γ^{tox-}), γ19, δ, (syn = δ^{tox+}), ρ, (syn = ρ^{tox-}), φ9, |
| | φ984, ω, 1A, 1/1180, 2, 2/1180, 5/1180, 5ad/9717, 7/4465, |
| | 8/4465, 8ad/10269, 10/9253, 13/9253, 15/3148, 21/9253, 28, |
| | 29, 55, 2747, 2893, 4498, 5848. |
| Cyanobacteria | Phormidium, Synechococcus, [click to view list of |
| | cyanobacteria phages] |
| Cytophaga | NCMB384, NN-Cytophaga (1). |
| Dactylosporangium | 9-41A, (syn = A1), (syn = A1-Dat), |
| Dermatophilus | φDM1. |
| Desulfovibrio | NN-Desulfovibrio (1). |
| Enterobacter | C3, WS-EO20, WS-EP26, WS-EP28, φmp, 667/617, 886. |
| Enterobacteria | Enterobacter, Erwinia, Escherichia, Klebsiella, Levienea, |
| | Morganella, Proteus, Providencia, Salmonella, Serratia, |
| | Shigella, Yersinia. |
| Enterococcus | DS96, H24, M35, P3, P9, SB101, S2, 2BII, 5, 182a, 705, |
| | 873, 881, 940, 1051, 1057, 21096C, NN-Enterococcus (1). |
| Erwinia | 59, 62, 843/60. |
| Erysipelothrix | NN-Erysipelothrix (1). |
| Escherichia | AC30, CVX-5, C1, DDUP, EC1, EC2, E21, E29, F1, F26S, |
| | F27S, Hi, HK022, HK97, (syn = ΦHK97), HK139, HK253, |
| | HK256, K7, ND-1, no.D, PA-2, q, S2, T1, (syn = α), (syn = P28), |
| | (syn = T-1), (syn = T₁), T3C, T5, (syn = T-5), (syn = T₅), |
| | UC-1, w, β4, γ2, λ, (syn = lambda), (syn = Φλ), ΦD326, φγ, |
| | Φ06, Φ7, Φ10, φ80, χ, (syn = χ₁), (syn = φχ), (syn = φχ₁), 2, 4, |
| | 4A, 6, 8A, 102, 150, 168, 174, 3000. |
| Eubacterium | NN-Eubacterium (1). |
| Flavobacterium | CMF-1-F, (syn = cmf-1-F), (syn = cM-φ1), (syn = ChMF-1-P), |
| | O6N-12P, O6N-24P, ΦMT5. |
| Fusobacterium | fv2377, fv2527, fv8501. |
| Haemophilus | N3. |
| Halobacterium | Hh-1, Hh-3, Ja1, S45, ΦN. |
| Halomonas | F5-4, F9-11, F12-9. |
| Helicobacter | HP1, NN-Helicobacter (1). |
| Hyphomicrobium | Hyfa-5, Hyfa-6, Hyfa-7, Hyfa-13, Hyfa-14, Hyfa-15, Hyfa-16, |
| | Hyfa-17, Hyfa-18, Hyfa-19, Hyfa-20, Hyfa-21, Hyfa-22, |
| | Hyfa-23, Hyfa-24, Hyfa-25, Hyfa-26, Hyfa-27, Hyfa-28, |
| | Hyfa-29, Hyfa-30, Hyfa-31, Hyfa-32, Hyfa-33, Hyfa-34, |
| | Hyfa-35, Hyfa-36, Hyfa-37, Hyfa-48, Hy-11, Hyφ32a. |
| Klebsiella | FC3-11, Kl₂B, (syn = Kl2B), Kl₂₅, (syn = Kl25), Kl₄₂B, (syn = Kl42), |
| | (syn = Kl42B), Kl₁₈₁B, (syn = Kl181), (syn = Kl181B), |
| | Kl_{765/1}, (syn = Kl765/1), Kl₈₄₂B, (syn = Kl832B), Kl₉₃₇B, (syn = Kl937B), |
| | L1, φ28, 7, 231, 483, 490, 632. |
| Lactobacillus | BA, BaF1, BU77-B1, B2, ch2, c3, (syn = c31), C-5, c5, c5h, |
| | F1, G, G₁₀, J1, (syn = J-1), (syn = J₁), LB₁, lb4, lb5, lb6, |
| | lb539, LC-Nu, LL-H, (syn = ll-h), (syn = φLL-H), LL-K, LL- |
| | Ku, LL-S, lv, NHs, PB, PH, PLS-1, PL-1, PL-2, PWH2, Sa- |
| | S, S-9, S171, UZ, y5, Z63-B2, φadh, φFSV, φFSW, φg1e, |
| | Φlh60, ΦLP1-A, ΦLP1-B, ΦLP2, ΦLP571, ΦLP651, φ41k, |
| | φ219, φ392-A2, φ393, φ786, 010, 011, 050R (defective), 056 |
| | (defective), 0237, 0252, 0448, (syn = mv4), 0449, (syn = mv1), |
| | 0494, 01013, 01014, 01243, 3-793, 11, 13, 15, 19, 112, |
| | 208 (defective), 227, 249R (defective), 432 (defective), 436 |
| | (defective), 535/222a, Ia11, II-5, NN-Lactobacillus (8). |
| Lactococcus | AC1L16M, A56-1, A69-4, B, br, (syn = φbr), BK5-T, (syn = BK5), |
| | (syn = BK5-T), (syn = ΦTBK5), B11-1, B39-1, c, C2, |
| | c2t₁, c2t₂, c3, C5W9, c11, c13, C36-3, C60-2, d, drc2, (syn = DRC₂), |
| | drc3, (syn = DRC₃), eb4, eb9, e8, E10-1, FRC2, |
| | FRC4, F4-1, F29-1, G69-1, G72-1, hp, I8, I37-1, I52, I66, |
| | I119, I129, jj18, jw1, jw2, jw4, jw8, jw9a, jw12, jw13, jw14, |
| | jw16B, jw25, jw27A, jw27, jw31, jw32, J29-1, K, LC1, |
| | LC2, LC3, (syn = φLC3), LC4, LC5, LC6, L11, L12, L13, ml1, |
| | m12r, (syn = φm12r), ot, (syn = φot), P, PLgY no. 4, PLgY |
| | no. 7, PLgY no. 16, PLgY no. 22, P002, P003, P008, P008S, |
| | P010, P013, P026, P031, P039, P047, P053, P059, P087, |
| | P096, p2, P107, P112ag, P113G, P114-4BN, B123BN, P142, |
| | P179, P191, P204, P219, P221BN, P228, P232, P239, P272, |
| | P315, P323, P335, R-I, R₁-T, (syn = R1-T), (syn = r₁t), (syn = Φr1t), |
| | r1v, r6, sg1, (syn = φsg1), sl122, sl123, sk1, (syn = Φsk1), |
| | TP-Bus3018, TP-Bus3021, TP-Bu2-K5, TP-C10, TP- |
| | J34, (syn = ΦTP-J34), (syn = φTP-J34), TP-P2/1-3, TP-Wis3-1, |
| | TP-Wis98.1, TPW22, TP-11-13, TP-21-2, TP-40-3, TP-712, |
| | TP-901-1, (syn = ΦTP901-1), TP-918, TP-936-1, TP-938-2, |
| | TP-951-1, TP3106, TP3107, Tuc2009, T104, uc311a, uc311b, |
| | uc311c, uc311d, uc311e, uc311f, uc411a, uc411b, uc411c, |
| | uc411d, uc411e, uc411f, uc412a, uc412b, uc412c, uc412d, |
| | uc412e, uc412f, uc450a, uc450b, uc450c, uc450d, uc1001, |
| | uc1002, UL4, UL7, UL9, UL10, UL11, UL12, UL13, UL14, |
| | UL22, UL23, UL35, UL36, (syn = ul36), ul37, w401c, |
| | w401t, w401x1, w406, w407c, w407t, w407x1, w411c, w411t, |
| | w501, w502, w503c, w503t, z8, φK27, φK70, φLC3, φMu1, |
| | φQ13, φQ30, φUS3, φ31, φ48, φ50, φ105-5, φ105-10, φ108, |
| | φ109, φ110, φ111, φ112, φ113, φ336-11, φ368, φ404, φ624, |
| | φ630, Φ779, φ783, φ806, φ815, φ825, φ886, φ927, Φ943, |
| | φ957, φ1002, φ1033, φ1034, φ1050, φ1076, φ1090, φ1094, |
| | φ1095, φ1097, φ1100, φ1190, φ1199, φ1256, 05, (syn = φ05), |
| | 10n, 10p, 18-6, 26-2, 28, 134-T, 187, (syn = φT187), 188, 189, |
| | 264, 265, 280A, 280B, 293, 601, 669, 670, 754, 776, 785, |
| | 799, 819, 838, 844, 845, 847, 852, (syn = φ852), 853, (syn = φ853), |
| | 855, 856, 859, 874, 876, (syn = φ876), 877, 878, (syn = φ878), |
| | 880, 881, 884, 889, 890, 891, 892, 893, 895, 896, |
| | 897, (syn = φ897), 898, 899, (syn = φ899), 900, 902 903, 904, |
| | 905, 907, 908, 909, 911, 912, 914, 915, 916 924, (syn = φ924), |
| | 925, 929, 933, 934, 935, 936, (syn = φ936), 937, 938, |
| | 939, (syn = φ939), 942, 944, 946, 946B, 947, 948, 949, (syn = φ949), |
| | 950, 951, 952, 953, 954, 955, 956, (syn = φ956), 958, |
| | (syn = φ958), 959, 963, 964B, 965, 966, 969, 970, 971, 972, |
| | 976, 977, 981, 982, 984, 985, 986, 990, 992, 993, 994, 995, |
| | 996, 998, 999, 1007, 1009, 1011, 1035, 1204, 1250, 1277, |
| | 1283, 1299, 1358, 1364, 1367, 1370, 1374, 1404, 1405, NN- |
| | Lactococcus (51). |
| Leuconostoc | fOg29, fOg30, fOg44, L1(Ia7), L2(Ia8), L3(Ea3), L4(Aa1), |
| | L5(Bb4), L6(ML34), L7(ML34), L8(1890), L9(Aa1), |
| | L10(Bb4), L11(Cb3), L12(Fa6), L13(Fb2), L14(Ib10), |
| | L15(Ib8), L16(MLS4), L17(Psu-1), L18(1674), L19(1890), |
| | L20(2119), LTH24P, LTH25P, LTH26P, LTH27P, LTH28P, |
| | LTH29P, LTH30P, LTH31P, LTH32P, LTH33P, LTH34P, |
| | ML34, POF025, pro, PSU₁, P58I, P58II, φcc2b, φcc5a, |
| | φcc59a, φcc59b, φcc62, φLo22a, φLo27a, φLo27b, φ335, |
| | φ336, φ399, φ400, Φ1002, ΦLco23, 1920, 1931, 1932, 4029, |
| | 5194, NN-Leuconostoc (6). |
| Levinea | DM-21. |
| Listeria | A005, A006, A020, A500, A502, A511, A118, A620, A640, |
| | B012, B021, B024, B025, B035, B051, B053, B054, B055, |
| | B056, B101, B110, B545, B604, B653, C707, D441, HSO47, |
| | H1OG, H8/73, H19, H21, H43, H46, H107, H108, H110, |
| | H163/84, H312, H340, H387, H391/73, H684/74, H924A, |
| | PSA, U153, φMLUP5, (syn = P35), 00241, 00611, 02971A, |
| | 02971C, 5/476, 5/911, 5/939, 5/11302, 5/11605, 5/11704, 184, |
| | 575, 633, 699/694, 744, 900, 1090, 1317, 1444, 1652, 1806, |
| | 1807, 1921/959, 1921/11367, 1921/11500, 1921/11566, |
| | 1921/12460, 1921/12582, 1967, 2389, 2425, 2671, 2685, 3274, |
| | 3550, 3551, 3552, 4276, 4277, 4292, 4477, 5337, |
| | 5348/11363, 5348/11646, 5348/12430, 5348/12434, 10072, |
| | 11355C, 11711A, 12029, 12981, 13441, 90666, 90816, |
| | 93253, 907515, 910716, NN-Listeria (15). |
| Methanobacterium | φF3, ψM1. |
| Methanobrevibacter | PG. |
| Micrococcus | N1, N2, N3, N4, N5, N6, N7, N8, sm26, sm59, W, X. |
| Micromonospora | MMP1, φUW21, φUW51. |
| Micropolyspora | φ-150A. |
| Microtetraspora | Mtc1. |
| Morganella | 47. |
| Mycobacterium | AG1, AL₁, ATCC 11759, A2, B.C₃, BG2, BK1, BK₅, |
| | butyricum, B-1, B5, B7, B30, B35, Clark, C1, C2, DNAIII, |
| | DSP₁, D4, D29, GS4E, (syn = GS₄E), GS7, (syn = GS-7), |
| | (syn = GS₇), IPα, lacticola, Legendre, Leo, L5, (syn = ΦL-5), |
| | MC-1, MC-3, MC-4, minetti, MTPH11, Mx4, MyF₃P/59a, |
| | phlei, (syn = phlei 1), phlei 4, Polonus II, rabinovitschi, |
| | smegmatis, TM4, TM9, TM10, TM20, Y7, Y10, φ630, 1B, |
| | 1F, 1H, 1/1, 67, 106, 1430. |
| Mycoplasma | NN-Mycoplasma (1). |
| Nocardia | MNP8, NJ-L, NS-8, N5, NN-Nocardia (1). |
| Nocardioides | X2, X6. |
| Nocardiopsis | φAC2. |
| Oerskovia | O2. |
| Paenibacillus | BA-4, BP52, BP142, BP153, FPy-1, IPy-1, NN-Paenibacillus |
| | (2). |
| Pasteurella | B932a, C-2, φPhA1, 32, 53, 115, 967, 1075. |
| Pediococcus | pa40, pa42, pa97. |
| Phormidium | NN-Phormidium (1). |
| Promicromonospora | P1. |
| Propionibacterium | P-a-1, P-a-2, P-a-3, P-a-4, P-a-5, P-a-6, P-a-7, P-a-8, P-a-9, |
| | PB2, TL110B7, NN-Propionibacterium (19). |
| Proteus | Clichy 12, π2600, φχ7, 1/1004, 5/742, 9, 12, 14, 22, 24/860, |
| | 2600/D52. |
| Providencia | 7/R49, 7476/322, 7478/325, 7479, 7480, 9000/9402, |
| | 9213/9211a. |
| Pseudoalteromonas | H103-1, H105-1, H108, H118/1, H120/1, 10-77a, 11-68c. |
| Pseudomonas | af, A7, B3, B33, B39, BI-1, C22, D3, D37, D40, D62, |
| | D3112, F7, F10, g, gd, ge, gf, Hw12, Jb19, KF1, L°, OXN- |
| | 32P, O6N-52P, PCH-1, PC13-1, PC35-1, PH2, PH51, PH93, |
| | PH132, PMW, PM13, PM57, PM61, PM62, PM63, PM69, |
| | PM105, PM113, PM681, PM682, PO4, PP1, PP4, PP5, PP64, |
| | PP65, PP66, PP71, PP86, PP88, PP92, PP401, PP711, |
| | PP891, Pssy41, Pssy42, Pssy403, Pssy404, Pssy420, Pssy923, |
| | PS4, PS-10, Pz, SD1, SL1, SL3, SL5, SM, φC5, φC11, |
| | φC11-1, φC13, φC15, φMO, φX, φ04, φ11, φ240, 2, 2F, 5, |
| | 7m, 11, 13, 13/441, 14, 20, 24, 40, 45, 49, 61, 73, 148, 160, |
| | 198, 218, 222, 236, 242, 246, 249, 258, 269, 295, 297, 309, |
| | 318, 342, 350, 351, 357-1, 400-1, NN-Pseudomonas (6). |
| Rhizobia | b, C1, d, h, i, JRW3, K1, K2, L412a, L422, L422a, L425a, |
| | L426a, L431, L434a, L439, L441, L449, M1, NM1, NM2, |
| | NM3, NM4, NM6, NM7, NM8, NT1, NT2, NT3, NT4, RC1, |
| | RC5, RL1, RS1, ΦA161, ΦFM1, Φgor3V, ΦLS5B, ΦMI-5, |
| | ΦM5N1, ΦM11S, ΦM14S, ΦM20S, ΦM23S, ΦFM26S, |
| | ΦM27S, Φ2011, φ2048, 5, 16-2-4, 16-3, 16-6-12, 16-12-1, |
| | 16-22-2, 317. |
| Rhodobacter | Rφ-1, Rφ6, Rφ6P, φRsA. |
| Rhodococcus | MJP1, MJP20, MJP25, MNP1, MNP2, MNP7, R1, φC, φEC |
| Ruminococcus | NN-Ruminococcus (1). |
| Saccharomonospora | 119, NN-Saccharomonospora (1?). |
| Saccharopolyspora | G3, G4, G5, Mp1, P113, P517, Tm1, φC69, φFRa-A, φFRa- |
| | C, φFRa-E, φFRb-B, φFRb-D, φFRb-M, φFRb-O, φFRb-P, |
| | φFRG9, φFrv-J, φFrv-N, φFrv-S, φFrv-T, φFR13, φFR113, |
| | φFR114, φFR371, φFR747, φFR755R, φSaC1, (syn = φLig), |
| | φSaG1, φSaV1, (syn = φG3a1), (syn = φLiv), φSE6, 3, 31, 121, |
| | 1527. |
| Saccharothrix | W1 |
| Salmonella | c, C236, C557, C625, C966N, g, GV, G5, G173, h, IRA, |
| | Jersey, MB78, P22-1, P22-3, P22-12, Sab1, Sab2, Sab2, |
| | Sab4, San1, San2, San3, San4, San6, San7, San8, San9, |
| | San13, San14, San16, San18, San19, San20, San21, San22, |
| | San23, San24, San25, San26, SasL1, SasL2, SasL3, SasL4, |
| | SasL5, S1BL, SII, ViII, φ1, 1, 2, 3a, 3aI, 1010, NN- |
| | Salmonella (1). [click to view list of enteric phages] |
| Selenomonas | M1, NN-Selenomonas (1). |
| Serratia | BC, BT, CW2, CW3, CW4, CW5, L₁232, L₂232, L34, |
| | L.228, SLP, SMPA, V.43, σ, φCW1, ΦCP6-1, ΦCP6-2, |
| | ΦCP6-5, 3T, 5, 8, 9F, 10/1, 20E, 32/6, 34B, 34CT, 34P, 37, |
| | 41, 56, 56D, 56P, 60P, 61/6, 74/6, 76/4, 101/8900, 226, |
| | 227, 228, 229F, 286, 289, 290F, 512, 764a, 2847/10, |
| | 2847/10a. |
| Shigella | B11, DDVII, (syn = DD7), FS_{D2b}, (syn = W₂B), FS₂, (syn = F₂), |
| | (syn = F2), FS₄, (syn = F₄), (syn = F4), FS₅, (syn = F₅), (syn = F5), |
| | FS₉, (syn = F₉), (syn = F9), F11, P2-SO-S, SG₃₆, (syn = SO-36/G), |
| | (syn = G36), SG₃₂₀₄, (syn = SO-3204/G), SG₃₂₄₄, |
| | (syn = SO-3244/G), SH_{I}, (syn = HI), SH_{VII}, (syn = HVII), SH_{IX}, |
| | (syn = HIX), SH_{XI}, SH_{XII}, (syn = HXII), SKI, KI, (syn = S_{I}), |
| | (syn = SsI), SKVII, (syn = KVII), (syn = S_{VII}), (syn = SsVII), |
| | SKIX, (syn = KIX), (syn = S_{IX}), (syn = SsIX), SKXII, (syn = KXII), |
| | (syn = S_{XII}), (syn = SsXII), ST_{I}, ST_{III}, ST_{IV}, ST_{VI}, ST_{VII}, |
| | S70, S206, U2-SO-S, 3210-SO-S, 3859-SO-S, 4020-SO-S, |
| | φ3, φ5, φ7, φ8, φ9, φ10, φ11, φ13, φ14, φ18. |
| Sphingomonas | T-φDO. |
| Spiroplasma | SV-C2. |
| Staphylococcus | AC1, AC2, A6″C″, A9″C″, b⁵⁸¹, CA-1, CA-2, CA-3, CA-4, |
| | CA-5, D11, L39x35, L54a, M42, N1, N2, N3, N4, N5, N7, |
| | N8, N10, N11, N12, N13, N14, N16, Ph6, Ph12, Ph14, UC- |
| | 18, U4, U15, S1, S2, S3, S4, S5, X2, Z1, φB5-2, φD, ω, 11, |
| | (syn = φ11), (syn = P11-M15), 15, 28, 28A, 29, 31, 31B, 37, |
| | 42D, (syn = P42D), 44A, 48, 51, 52, 52A, (syn = P52A), 52B, |
| | 53, 55, 69, 71, (syn = P71), 71A, 72, 75, 76, 77, 79, 80, |
| | 80α, 82, 82A, 83A, 84, 85, 86, 88, 88A, 89, 90, 92, 95, 96, |
| | 102, 107, 108, 111, 129-26, 130, 130A, 155, 157, 157A, |
| | 165, 187, 275, 275A, 275B, 356, 456, 459, 471, 471A, 489, |
| | 581, 676, 898, 1139, 1154A, 1259, 1314, 1380, 1405, 1563, |
| | 2148, 2638A, 2638B, 2638C, 2731, 2792A, 2792B, 2818, |
| | 2835, 2848A, 3619, 5841, 12100. |
| Streptococcus | AT298, A5, a10/J1, a10/J2, a10/J5, a10/J9, A25, BT11, b6, |
| | CA1, c20-1, c20-2, DP-1, Dp-4, DT1, ET42, e10, F_{A}101, |
| | F_{E}Ths, F_{K}, F_{KK}101, F_{KL}10, F_{KP}74, F_{K}11, F_{LO}Ths, F_{Y}101, f1, |
| | F₁₀, F₂₀140/76, g, GT-234, HB3, (syn = HB-3), HB-623, HB- |
| | 746, M102, O1205, φO1205, PST, P0, P1, P2, P3, P5, P6, |
| | P8, P9, P9, P12, P13, P14, P49, P50, P51, P52, P53, P54, |
| | P55, P56, P57, P58, P59, P64, P67, P69, P71, P73, P75, |
| | P76, P77, P82, P83, P88, sc, sch, sf, Sfi11, (syn = SFi11), |
| | (syn = φSFi11), (syn = ΦSfi11), (syn = φSfi11), sfi19, (syn = SFi19), |
| | (syn = φSFi19), (syn = φSfi19), Sfi21, (syn = SFi21), |
| | (syn = φSFi21), (syn = φSfi21), ST_{G}, STX, st2, ST₂, ST₄, S3, |
| | (syn = φS3), s265, Φ17, φ42, Φ57, φ80, φ81, φ82, φ83, φ84, |
| | φ85, φ86, φ87, φ88, φ89, φ90, φ91, φ92, φ93, φ94, φ95, φ96, |
| | φ97, φ98, φ99, φ100, φ101, φ102, φ227, Φ7201, ω1, ω2, |
| | ω3, ω4, ω5, ω6, ω8, ω10, 1, 6, 9, 10F, 12/12, 14, 17SR, |
| | 19S, 24, 50/33, 50/34, 55/14, 55/15, 70/35, 70/36, 71/ST15, |
| | 71/45, 71/46, 74F, 79/37, 79/38, 80/J4, 80/J9, 80/ST16, |
| | 80/15, 80/47, 80/48, 101, 103/39, 103/40, 121/41, 121/42, |
| | 123/43, 123/44, 124/44, 337/ST17, NN-Streptococcus (34). |
| Streptomyces | A, AP-3, AP-2863, Bα, B-I, B-II, CPC, CPT, CT, CTK, |
| | CWK, ES, FP22, FP43, K, MSP4, MSP7, MSP10, MSP11, |
| | MSP15, MSP16, MSP17, MSP18, MSP19, MVP4, MVP5, |
| | P8, P9, P13, P23, RP2, RP3, RP10, R₁, R4, SAP1, SAP2, |
| | SAP3, SAt1, SA6, SA7, SC1, SH10, SL1, SV2, TG1, type |
| | Ia, type II, type V, VC11, VP1, VP5, VP7, VP11, VWB, |
| | VW3, WSP3, φA1, φA2, φA3, φA4, φA5, φA6, φA7, φA8, |
| | φA9, φBP1, φBP2, φC31, (syn = φc31), (syn = φ31C), (syn = C31), |
| | φC43, φHAU3, φSF1, φSPK1, 4, 5a, 5b, 8, 10, 12b, |
| | 13, 17, 19, 22, 23, 25, 506, NN-Streptomyces(3). |
| Synechococcus | S-BBS1, SM-2, S-1, S-2L(L), (syn = S-2L), S-4L(L), (syn = S- |
| | 4L). |
| Tetragenococcus | φD, φD-10. |
| Thermoactinomyces | φ-115A. |
| Thermomonospora | Tb1, Tf2, Tf3, Tf4, Thf2, Thf3, NN-Thermomonospora (1?). |
| Thermopseudosporangium | Tsp8, Tsp10, Tsp38. |
| Treponema | NN-Treponema (1). |
| Veillonella | N20, N40. |
| Vibrio | hv-1, OXN-52P, P13, P38, P53, P65, P108, P111, TP1, VP3, |
| | VP6, VP12, VP13, 70A-3, 70A-4, 70A-10, 72A-1, 108A-3, |
| | 109-B1, 110A-2, 149, (syn = φ149), IV, (syn = group IV), NN- |
| | Vibrio (22). |
| Xanthomonas | A342, HXX, PG60, P1-3a, P6, XO3, XO4, XO5, 20, 22, |
| | NN-Xanthomonas (1). |
| Xenorhabdus | NN-Xenorhabdus (1). |
| Yersinia | Yer2AT. |

SIPHOVIRIDAE, morphotype B2
tail long and noncontractile, head elongated (length/width ratio = 1.2-2)

| | |
|---|---|
| Acinetobacter | A19, A23, A29, B₉PP, 59, 531, 2845. |
| Agrobacterium | GP1, PT11, SL4, SL6, SL7, SW7, SW12, SW14. |
| Asticcacaulis | φAcM₂, φAcM₃, φAcM₄, φAcM₅. |
| Bacillus | Bat10, BSL10, BSL11, BS6, BS11, BS16, BS23, BS101, BS102, |
| | g18, mor1, PBL1, SN45, thu2, thu3, Tm1, Tm2, TP-20, TP21, |
| | TP52, type F, type G, type IV, NN-Bacillus (3). |
| Caryophanon | φCVL-29. |
| Caulobacter | φ6, 76. |
| Clostridium | CA1, F1, K, S2, 1, 5, NN-Clostridium (8). |
| Enterobacteria | Escherichia, Klebsiella, Proteus, Salmonella, Serratia, Shigella. |
| Enterococcus | PE1 |
| Escherichia | AC6, AC7, AC28, AC43, AC50, AC57, AC81, AC95, HK243, |
| | K10, ZG/3A, 5, 5A, 21EL. |
| Hyphomicrobium | Hy-39, Hy-40, Hy-41, Hy-42. |
| Klebsiella | 864/100. |
| Lactobacillus | 223, 050E (defective), 249E (defective). |
| Lactococcus | a, b, bIL67, c1, c2, c6A, c10I, c10II, drc1, drc3, D59-1, e, eb7, |
| | E16-2, FRC1, FRC3, h1, I16-1, KC1, P001, P029, P6, P25, P42, |
| | P67, P109, P127, P159, P167, P177, P188, P220, P330, R₁, |
| | sl23, STl1, Stl3, Stl5, T₂₄, uc1003, UL1, UL2, UL3, UL6, |
| | UL15, UL16, UL17, UL19, UL21, UL24, UL25, UL26, UL28, |
| | UL29, UL30, UL31, UL38, φQ38, Φ1Sᵥ, Φ2Sᵥ, Φ3Sᵥ, Φ4Sₜ, |
| | Φ6Sₜ, Φ12Sᵥ, Φ13Sᵥ, φ152, φ172, 3ML, (syn = ML3), (syn = ml3), |
| | 10w, 25, 106, 160, 296, 335, 917, 643, (syn = φ643), 690, |
| | (syn = φ690), 779, (syn = φ779), 842, 879, 917, 920, 921, 923, |
| | (syn = φ923), 928, 943, 961, 964, 964A, 967, 968, 979, 991 |
| | 1039, 1045, 1046, 1056, 1061, 1064, 1070, 1072, 1123, 1124, |
| | 1143, 1146, 1174, 1175, 1176, 1177, 1178, (syn = φ1178), 1280, |
| | (syn = φ1280), 1310, 1337, 1378, NN-Lactococcus (2). |
| Mycobacterium | B1, (syn = Bo1), B₂₄, D, D29, F-K, F-S, HP, Polonus I, Roy, R1, |
| | (syn = R1-Myb), (syn = R₁), 11, 31, 40, 50, 103a, 103b, 128, |
| | 3111-D, 3215-D, NN-Mycobacterium (1). |
| Proteus | Pm8, ²⁴/₂₅14. |
| Pseudomonas | G101, M6, M6a, L1, PB2, Pssy15, Pssy4210, Pssy4220, PYO12, |
| | PYO34, PYO49, PYO50, PYO51, PYO52, PYO53, PYO57, |
| | PYO59, PYO200, PX2, PX5, SL4, φ03, φ06, 1214. |
| Rhizobia | F4/L425I, F4/L425II, F5, F5/L422, H3V, L419, L432a, NM5, |
| | Φ1261M, (syn = φgal1261/M), Φ1261V, φ2037/1, φ2037/2, |
| | φ2037/3, φ2037/4, φ2037/5, φ2037/6, φ2037/7, φ2205, 1, NN- |
| | Rhizobia (2). |
| Rhodobacter | RZ1, φRsG1, (syn = ΦRsG1), |
| Salmonella | N-4, SasL6, 27. |
| Serratia | L.359, SMB1. |
| Shigella | SH_{III}, (syn = HIII), SH_{XI}, (syn = HXI), SK_{XI}, (syn = KXI), (syn = S_{XI}), |
| | (syn = SsXI), (syn = XI). |
| Sphaerotilus | SN₁. |
| Staphylococcus | AC3, A8, A10, A13, b594n, D, HK2, N9, N15, P52, P87, S1, |
| | S6, Z₄, φRE, 3A, 3B, 3C, 6, 7, 16, 21, 42B, 42C, 42E, 44, 47, |
| | 47A, 47C, 51, 54, 54x1, 70, 73, 75, 78, 81, 82, 88, 93, 94, |
| | 101, 105, 110, 115, 129/16, 174, 594n, 1363/14, 2460, NN- |
| | Staphylococcus (1). |
| Streptomyces | Mex, MLSP1, MLSP2, MLSP3, MSP1, MSP2, MSP3, MSP5, |
| | MSP8, MSP9, MSP12, MSP13, MSP14, R2, SA1, SA2, SA3, |
| | SA4, SA5, type I, type Ia, (syn = 35/35), type III, type IV, WSP1, |
| | WSP4, WSP5, 2b, 4, 15, (syn = C), 26, 8238. |
| Thermoactinomyces | M1, M3. |
| Vibrio | VP5, VP11, VP15, VP16, α1, α2, α3a, α3b, 353B, NN-Vibrio |
| | (7). |
| Xanthomonas | XP12, (syn = XP-12), (syn = Xp12), φPS, φRS, φSD, φSL, φ56, |
| | φ112, 1. |

SIPHOVIRIDAE, morphotype B3
tail long and noncontractile, head elongated
(length/width ratio = 2.5 or more)

| | |
|---|---|
| Aneurinobacillus | NN-Aneurinobacillus (1). |
| Asticcacaulis | φAcS₁, φAcS₂, φAc41. |
| Bacillus | BLE, (syn = θc), BS2, BS4, BS5, BS7, B10, B12, BS20, BS21, |
| | F, MJ-4, PBA12. |
| Caulobacter | φCbK, φCb3, φCb6, φCb13, φCp34, φCr2, φCr4, φCr5, φCr6, |
| | φCr7, φCr8, φCr9, φCr10, φCr11, φCr12, φCr13, φCr15, φCr20, |
| | φCr21, φCr23, φCr25, φCr27, φCr29, φCr31, φCr32, φCr33, |
| | φCr34, φCr36, φCr37, φCr38, φCr39, φCr42, φCr43. |
| Enterococcus | F1, F3, F4, VD13, 1, 200, 235, 341. |
| Escherichia | H19-J, 933H. |
| Lactobacillus | JCL1032, 235, φy8, NN-Lactobacillus (1). |
| Rhizobia | 7-7-7. |

TECTIVIRIDAE
icosahedral capsid with inner lipoprotein vesicle, linear dsDNA, “tail” produced for DNA injection

| | |
|---|---|
| Alicyclobacillus | A, φNS11 |
| Bacillus | AP50, AP50-04, AP50-11, AP50-23, AP50-26, AP50-27, |
| | Bam35. |
| Enterobacteria- | L172, PRD1, PR3, PR4, PR5, PR772 |
| Pseudomonas | |
| Thermus | P37-14. |

Miscellaneous Phages

| Pseudoalteromonas | PM2. |
|---|---|
| Pseudomonas | f6. |
| Acidianus | DAV1. |
| Haloarcula | His |
| Sulfolobus | NDV, SSV1, (syn = SAV1), SSV2, SSV3, SSVx. |
| Sulfolobus | SIRV, (syn = SIRV-2). |
| Key to Table 1: | |
| Left hand column contains the genus name of the bacterial host(s) which can be infected by the phages listed in the right hand column. Groups of phages are organized by family name and/or taxonomic criteria adopted bythe Ecology Phage group. Family name of Phages are underlined, and followed in the next line by a brief structural | |
| # description of the family members. Members of the underlined family immediately follow the structural description. | |

Source: Bacteriophage Names 2000, on the Bacteriophage Ecology Group Web site, hosted by the Ohio State University, Mansfield campus web site.

1. A method of detecting a bacterial infection in a subject, the method comprising:
administering to a subject an effective dose of labeled bacteriophage; and
imaging the labeled bacteriophage in a portion of the subject;
whereby the presence of labeled bacteriophage indicates the presence of a bacterial infection.

2. The method of claim 1, wherein the absence of labeled bacteriophage indicates a non-bacterial inflammation.

3. The method of claim 1, wherein the imaged portion of the subject comprises a location of a suspected or diagnosed inflammation.

4. The method of claim 3, further comprising:
comparing the level of labeled bacteriophage that localizes to the location of the suspected or diagnosed inflammation to a control level;
whereby a level of bacteriophage at the location of suspected or diagnosed inflammation that is above the control level indicates a bacterial infection.

5. The method of claim 4, wherein the control level is the background level of labeled bacteriophage that localizes to a portion of the subject that does not comprise a location of a suspected or diagnosed inflammation.

6. The method of claim 5, wherein the control level is provided by a protocol for diagnosing a bacterial infection.

7. A method of treating an inflammation in a subject, the method comprising:
performing the method of claim 1, wherein the presence of labeled bacteriophage at the site of suspected or diagnosed inflammation indicates a bacterial infection, and
subsequently treating the subject with an effective amount of treatment for the bacterial infection.

8. A method of treating an inflammation in a subject, the method comprising:
performing the method of claim 2, wherein the absence of labeled bacteriophage at the site of suspected or diagnosed inflammation indicates a non-bacterial inflammation, and subsequently treating the subject with an effective amount of treatment for a non-bacterial inflammation.

9. A method of identifying a type of bacterial infection in a subject, the method comprising:
administering to a subject an effective dose of at least a first type of labeled bacteriophage that is specific for one or more first bacterial strains or species;
imaging a portion of the subject; and
evaluating a level of at least one of the administered labeled bacteriophages in the imaged portion of the subject;
whereby a level of labeled bacteriophage above a control level in the imaged portion of the subject indicates the presence of one or more first bacterial strains or species.

10. The method of claim 9, wherein the evaluated level of bacteriophage indicates that the subject is not infected by a first bacterial strain or species for which the first bacteriophage or bacteriophages are specific, and the method further comprises:
administering to the subject an effective amount of at least one second type of labeled bacteriophage that is specific for one or more second bacterial strains or species different than the first type of labeled bacteriophage or labeled bacteriophages;
imaging a portion of the subject; and
evaluating the level of at least one of the second type of labeled bacteriophage;
whereby a level of the second type of labeled bacteriophage above a control level in the imaged portion of the subject indicates the presence of one or more of the different second bacterial strains or species.

11. The method of claim 9, further comprising:
administering to the subject a cocktail comprising effective amounts of each of two or more labeled types of bacteriophage, wherein each type of bacteriophage exhibits a different range of bacterial host specificity;
imaging a portion of the subject; and
evaluating the level of at least one type of administered labeled bacteriophage;
whereby a level of labeled bacteriophage above a control level in the imaged portion of the subject indicates an infection by the bacterial host of the labeled bacteriophage.

12. The method of claim 11, wherein each type of bacteriophage is differently labeled.

13. A method of diagnosing and treating an inflammatory response, the method comprising:
performing the method of claim 9, wherein the level of at least one labeled bacteriophage at the site of suspected or diagnosed inflammation indicates a bacterial infection; and
subsequently treating the subject with an effective amount of treatment for a bacterial infection.

14. A method of diagnosing and treating an inflammatory response, the method comprising:
performing the method of claim 9, wherein the level of at least one labeled bacteriophage at the site of suspected or diagnosed inflammation indicates a non-bacterial inflammation; and
subsequently treating the subject with an effective amount of treatment for a non-bacterial inflammation.

15. The method of claim 7, wherein the treatment comprises one or more of: ciprofloxacin, tetracycline, minocycline, doxycycline, erythromycin; clarithromycin, cephalosporins; amoxicillin; azithromycin; ofloxacin; ceftriaxone; and metronidazole.

16. The method of claim 8, wherein the treatment excludes treatment with an antibiotic.

17. The method of claim 1, further comprising performing a second imaging of the labeled bacteriophage in the portion of the subject.

18. The method of claim 17, further comprising administering to the subject a second dose comprising an effective amount of labeled bacteriophage prior to the second imaging.

19. The method of claim 17, further comprising evaluating the level of labeled bacteriophage after the first imaging and after the second imaging.

20. The method of claim 17, wherein a second dose of labeled bacteriophage is administered and the first dose of bacteriophage has a different label from the second dose of bacteriophage.

21. The method of claim 19, further comprising:
comparing the levels of bacteriophage from the first and second imagings of the subject to thereby track the course of a bacterial infection.

22. A method of treating a bacterial infection in a subject, the method comprising:
performing the method of claim 21, wherein the subject is treated for a bacterial infection after the first imaging, and the second imaging is performed after the treatment for a bacterial infection; and
adjusting the treatment for the bacterial infection based on a comparison of the levels of bacteria at the site of infection indicated by the first and second imagings.

23. A bacteriophage conjugated with mercaptoacetyl-triglycine (MAG₃).

24. The bacteriophage of claim 23, wherein the MAG₃ is chelated to a label.

25. The bacteriophage of claim 24, wherein the label is a radiolabel.

26. The bacteriophage of claim 25, wherein the radiolabel is ^{99m}Tc.

27. A bacteriophage radiolabeled with ^{99m}Tc.

28. A method of imaging a bacterial infection in a subject, the method comprising:
administering to a subject an effective dose of labeled bacteriophage; and
imaging the labeled bacteriophage in a portion of the subject.

29. The method of claim 28, wherein the label is selected from the group consisting of: a radiolabel, a fluorescent label, and a contrast agent.
